(19) United States
(12) Patent Application Publication
(10) Pub. No.: US 2026/0125371 A1
(43) Pub. Date: May 7, 2026

(54) METHOD FOR INHIBITING CLOSTRIDIOIDES DIFFICILE SPORE GERMINATION

(71) Applicant: UNIVERSITY OF NOTRE DAME DU LAC, South Bend, IN (US)

(72) Inventors: Mayland CHANG, Granger, IN (US); Shahriar MOBASHERY, Granger, IN (US); Yuanyuan QIAN, South Bend, IN (US)

(73) Assignee: UNIVERSITY OF NOTRE DAME DU LAC, South Bend, IN (US)

(21) Appl. No.: 19/119,981

(22) PCT Filed: Oct. 13, 2023
(86) PCT No.: PCT/US2023/076849

§ 371 Date: Apr. 10, 2025

Related U.S. Application Data
(60) Provisional application No. 63/415,872, filed on Oct. 13, 2022.

(51) Int. Cl. C07D 413/04; A61K 31/4245; A61K 31/4439; A61P 31/04; C07D 271/06

(52) U.S. Cl. CPC C07D 413/04; A61K 31/4245; A61K 31/4439; A61P 31/04; C07D 271/06

(57) ABSTRACT

Oxadiazole antibiotics that exhibit bactericidal activity against C. difficile vegetative cells. We screened a library of 75 oxadiazoles against C. difficile ATCC 43255. The findings from this collection served as the basis for the syntheses of an additional 58 analogs, which were tested against the same strain. We discovered a potent (MIC₅₀=0.5 μg/mL, MIC₉₀=1 μg/mL for 101 C. difficile strains) and narrow-spectrum oxadiazole (3-(4-(cyclopentyloxy)phenyl)-5-(4-nitro-1H-imidazol-2-yl)-1,2,4-oxadiazole; compound 57) that is not active against common gut bacteria or other tested organisms, but is selectively bactericidal against C. difficile and targets cell-wall synthesis. Other similarly effective oxadiazole antibiotics of formula I and II are described herein, several of which inhibit or prevent C. difficile spore germination.

### RELATED APPLICATIONS

This application claims priority under 35 U.S.C. § 119(e) to U.S. Provisional Patent Application No. 63/415,872, filed Oct. 13, 2022, which is incorporated herein by reference.

### BACKGROUND OF THE INVENTION

Infections caused by Clostridioides difficile (previously known as Clostridium difficile) are an urgent public health threat that resulted in 202,600 hospitalizations and 11,500 deaths in the United States in 2019. C. difficile is a Gram-positive anaerobic opportunistic bacterium, which colonizes the gut in patients who have used broad-spectrum antibiotics that disrupt the gut microflora. Damage to the gut epithelium from toxins produced by C. difficile results in inflammation and diarrhea. C. difficile infection (CDI) produces spores that can remain dormant for days/months and are not affected by antibiotics. Bile acids in the host gastrointestinal tract initiate germination of the spores, converting them into active vegetative cells, starting the cycles of re-infection. Recurrent CDI occurs in about 25% of patients. The current antibiotics for treatment of CDI are vancomycin (VAN), fidaxomicin (FDX), and metronidazole (MTZ), with the first two used as first-line treatments. FDX has a narrower spectrum of activity compared to VAN and MTZ, and this likely explains its lower 15% recurrence of infection, compared to 24% for VAN and 27% for MTZ. Antibiotics with narrow-spectrum activity that selectively target C. difficile would provide significant advantage to current treatments, as gut microflora dysbiosis that contributes to recurrence of CDI would be avoided.

Accordingly, new antibiotics are needed to reduce C. difficile vegetative cells, as well as inhibit toxins and spores, and at the same time would not encourage microbial resistance or affect the host microbiota. In addition, the antibiotic should not cause adverse events in the host. It is extremely challenging for an antibiotic to meet all these criteria, therefore new antibiotics for treating CDIs are urgently needed.

### SUMMARY

We previously reported (ACS Med. Chem. Lett. 2020, 11, 322) on the 1,2,4-oxadiazole class of antibacterials active against methicillin-resistant Staphylococcus aureus (MRSA). The oxadiazoles were discovered by in silico screening of 1.2 million compounds against penicillin-binding protein (PBP)2a, an essential enzyme whose catalytic activity confers resistance to β-lactam antibiotics. The lead oxadiazole (compound 1, ND-421) exhibits a minimal-inhibitory concentration (MIC) against MRSA of 2 μg/mL and is efficacious in mouse models of MRSA infection. We evaluated the activity of 1 against C. difficile ATCC 43255 strain and found a MIC of 4 μg/mL (Proc. Natl. Acad. Sci. USA. 2023, 120, e2304110120) and also reported on the discovery of oxadiazole 2, with a MIC of 2 μg/mL against C. difficile ATCC 43255.

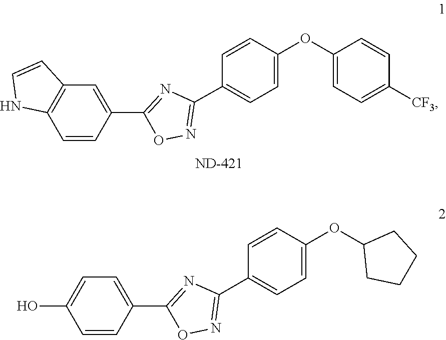 [SMILES: FC(F)(F)c1ccc(Oc2ccc(-c3noc(-c4ccc5[nH]ccc5c4)n3)cc2)cc1 ; Oc1ccc(-c2nc(-c3ccc(OC4CCCC4)cc3)no2)cc1]

We screened our existing library of 75 oxadiazoles, against C. difficile ATCC 43255 to develop preliminary structure-activity relationships (SAR) for this series. Building on the observed SAR for this study, we synthesized an additional 58 analogs for in vitro evaluation. As a result, we discovered a potent (MIC=0.25 μg/mL against C. difficile ATCC 43255) and narrow-spectrum oxadiazole (compound 57) that is not active against representative gut bacteria or other Gram-positive and Gram-negative bacteria. Compound 57 is bactericidal against vegetative C. difficile and targets cell-wall synthesis.

Accordingly, this disclosure provides a compound of formula I or II:

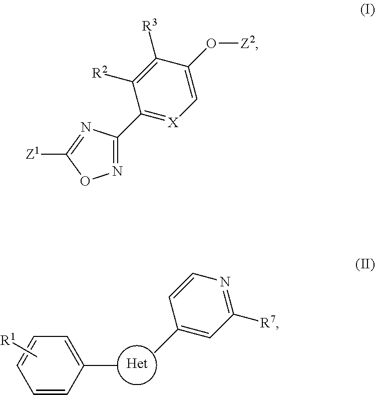

or a pharmaceutically acceptable salt thereof;
wherein,
Het is a 1,2,4-oxadiazole;
R¹ is aminoalkyl or OH; located ortho, meta, or para to Het;
R² is H, CF₃, NH₂, or 3-(trifluoromethyl)-3H-diazirine-3-yl;
R³ is H, CF₃, NH₂, or 3-(trifluoromethyl)-3H-diazirine-3-yl;
X is CH or N;
Z¹ is an imidazole, pyrazole, pyrrolidinone, phenyl, or an aminoalkyl, each optionally substituted (for example, nitro-imidazole, nitro-pyrazole, (aminoalkyl)phenyl, or hydroxylphenyl);
Z² is cyclopentyl or —(C₃-C₆)cycloalkyl, branched or unbranched —(C₁-C₆)alkyl, or Ar; and
R⁷ is Ar or OAr;
   wherein Ar is:

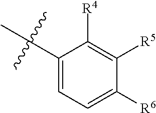

wherein R⁴, R⁵, and R⁶ are each independently H, halo (e.g., F or Cl), CH₂(halo), CF₃, —C(═O)CH₃, —C≡CH or NO₂;
further, wherein the variables of formulas I and II, and their sub-formulas III, IV, and V, can also be the substituents as illustrated for the corresponding variable for any compound of Charts 1, 2, and 3, hereinbelow, or a select subset thereof;
optionally, provided Z² is not cyclopentyl when Z¹ is 4-(aminomethyl)phenyl or 4-hydroxylphenyl, and/or optionally excluding any compound described by U.S. Pat. No. 11,168,062 (Chang et al.).

Additionally, a method is disclosed for treating a Clostridioides difficile infection (CDI) comprising administering to a subject having a CDI a therapeutically effective dose of a compound described herein, wherein the compound inhibits germination of a Clostridioides difficile spore that is present in the CDI and the subject is thereby treated.

The invention provides novel compounds of formula I-V, intermediates for the synthesis of compounds of formula I-V, as well as methods of preparing compounds of formula I-V. The invention also provides compounds of formula I-V that are useful as intermediates for the synthesis of other useful compounds. The invention provides for the use of compounds of formula I-V for the manufacture of medicaments useful for the treatment of bacterial infections in a mammal, such as a human.

The invention provides for the use of the compositions described herein for use in medical therapy. The medical therapy can be treating bacterial infections, for example, an infection by a gram-positive spore-forming anaerobic bacterium. The invention also provides for the use of a composition as described herein for the manufacture of a medicament to treat a bacterial infection in a mammal, for example, C. difficile infection in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. Antibacterial activity of oxadiazole 57 against C. difficile ATCC 43255. (A). Time-kill assay of 57, VAN, and MTZ at 8× MIC show 4-log 10 reduction in bacterial growth for oxadiazole 57 and MTZ. (B). PAE of oxadiazole 57, VAN, MTZ at 8× MIC; antibiotics were diluted 1000-fold after 1-h exposure. (C). Serial passage showed 8-fold increase in MIC for oxadiazole 57 (initial MIC of 0.25 μg/mL), 16-fold increase in MIC for VAN (initial MIC of 0.5 μg/mL), and 2-fold MIC increase for MTZ (initial MIC of 0.25 μg/mL).

FIG. 2A-C. Time-kill assay of oxadiazole 57 against C. difficile ATCC 43255 at (A) 1× MIC (0.25 μg/mL), (B) 2× MIC (0.5 μg/mL), and (C) 4× MIC (1 μg/mL). Bactericidal activity (≥3 log₁₀ reduction) is observed at 4× MIC for 12 h.

FIG. 3A-C. PAE of oxadiazole 57 against C. difficile ATCC 43255 vegetative cells at (A) 1× MIC (0.25 μg/mL), (B) 2× MIC (0.5 μg/mL), and (C) 4× MIC (1 μg/mL). Antibiotics were diluted 1000-fold after 1-h exposure.

FIG. 4. Observation of the effect of oxadiazole 57 by SEM. C. difficile ATCC 43255 was treated with antibiotic at 8× MIC for 24 h; a negative control (no treatment) was included. Scale bars are 1 μm. VAN and 57 damage the cell wall of vegetative cells, the outmost surface in Gram-positive bacteria.

### DETAILED DESCRIPTION

Clostridioides difficile is an anaerobic Gram-positive bacterium that colonizes the gut of patients treated with broad-spectrum antibiotics. The normal gut microflora prevents C. difficile colonization, however dysbiosis by treatment with broad-spectrum antibiotics causes recurrence of CDI in 25% of patients. There are no fully effective antibiotics for the treatment of multiple recurrent CDI. We report herein that oxadiazole antibiotics exhibit bactericidal activity against C. difficile vegetative cells. We screened a library of 75 oxadiazoles against C. difficile ATCC 43255. The findings from this collection served as the basis for the syntheses of an additional 58 analogs, which were tested against the same strain. We report a potent (MIC₅₀=0.5 μg/mL, MIC₉₀=1 μg/mL for 101 C. difficile strains) and narrow-spectrum oxadiazole (3-(4-(cyclopentyloxy)phenyl)-5-(4-nitro-1H-imidazol-2-yl)-1,2,4-oxadiazole; compound 57), which is not active against common gut bacteria or other tested organisms. Compound 57 is selectively bactericidal against C. difficile and targets cell-wall synthesis.

### Definitions.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability, necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) inhibiting the disease, pathologic or medical condition or arresting its development; (ii) relieving the disease, pathologic or medical condition; and/or (iii) diminishing symptoms associated with the disease, pathologic or medical condition. Thus, the terms “treat”, “treatment”, and “treating” include lowering, stopping, or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

In general, a “substituent” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, arylalkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SON(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (1-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7.

The term “alkoxy” refers to the group alkyl-O—, where alkyl is as defined herein. Examples of alkoxy groups include, but are not limited to, methoxy, ethoxy, n-propoxy, iso-propoxy, n-butoxy, tert-butoxy, sec-butoxy, n-pentoxy, n-hexoxy, 1,2-dimethylbutoxy, and the like. The alkoxy can be unsubstituted or substituted as described for alkyl groups.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R3N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is an independently selected substituent such as alkyl, optionally including protonated forms of each. Accordingly, any compound substituted with an amino group can be viewed as an amine.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted, as described for alkyl groups (below).

The term “heterocycle” refers to a saturated or partially unsaturated ring system, containing at least one heteroatom selected from the group oxygen, nitrogen, silicon, and sulfur, and optionally substituted with one or more groups as defined for the term “substituted”. A heterocycle can be a monocyclic, bicyclic, or tricyclic group. Such heterocycles may also be aromatic. Therefore, “heteroaryls” are a subset of heterocycles. A heterocycle group also can contain an oxo group (═O) or a thioxo (═S) group attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholinyl, piperazinyl, piperidinyl, pyrazolidinyl, pyrazolinyl, pyrrolidine, pyrroline, quinuclidine, tetrahydrofuranyl, and thiomorpholine.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein.

### Statements of the Technology.

1. A compound of formula I or II:

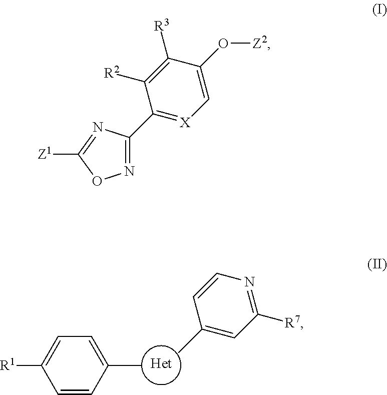

or a pharmaceutically acceptable salt thereof;
wherein,
   Het is a 1,2,4-oxadiazole;
   R¹ is aminoalkyl or OH;
   R² is H or NH₂;
   R³ is H, CF₃, or 3-(trifluoromethyl)-3H-diazirine-3-yl;
   X is CH or N;
   Z¹ is nitro-imidazole, nitro-pyrazole, pyrrolidinone, 4-(aminoalkyl)phenyl, 4-hydroxylphenyl, or aminoalkyl;
   Z² is cyclopentyl or —(C₃-C₄ or C₆)cycloalkyl, branched or unbranched —(C₁-C₆)alkyl, or Ar wherein Ar is:

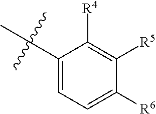

wherein,
   R⁴ is H, CH₂(halo), or NO₂;
   R⁵ is H or halo (e.g., F); and
   R⁶ is H, halo (e.g., F), CF₃, —C(═O)CH₃, or —C≡CH; and
R⁷ is Ar or OAr;
provided Z² is not cyclopentyl when Z¹ is 4-(aminomethyl)phenyl or 4-hydroxylphenyl.
2. The compound of embodiment 1 wherein Z¹ is 5-nitro-1H-imidazole-2-yl, —(CH₂)₄NH₂, 4-hydroxyphenyl, 4-(NH₂CH₂)phenyl, 4-(CH₃NHCH₂)phenyl, 4-nitro-1H-pyrazole-3-yl, or pyrrolidin-2-one-4-yl.
3. The compound of embodiment 1 or 2 wherein Z² is cyclopentyl, cyclopropyl, cyclobutyl, or cyclohexyl.
4. The compound of embodiment 1 wherein the compound is 57:

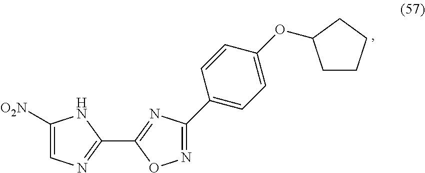 [SMILES: O=[N+]([O-])c1cnc(-c2nc(-c3ccc(OC4CCCC4)cc3)no2)[nH]1]

a pharmaceutically acceptable salt thereof.
5. The compound of any one of embodiments 1-3 wherein Z² is 4-(trifluoromethyl)phenyl, 4-fluorophenyl, 3,4-difluorophenyl, 4-iodophenyl, 3-iodophenyl, 2-nitrophenyl, 2-(bromomethyl)phenyl, phenyl, 4-acetophenyl, or 4-phenylethyne.
6. The compound of embodiment 1 wherein formula I is represented by formula III:

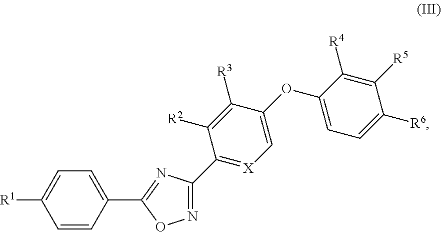

or
a pharmaceutically acceptable salt thereof;
wherein,
   R¹ is aminoalkyl or OH;
   R² is H or NH₂;
   R³ is H, CF₃, or 3-(trifluoromethyl)-3H-diazirine-3-yl;
   R⁴ is H, —CH₂(halo), or NO₂;
   R⁵ is H or halo;
   R⁶ is H, halo, CF₃, —C(═O)CH₃, or —C≡CH; and
   X is CH or N.
7. The compound of embodiment 6 wherein R¹ is —CH₂NH₂, —CH₂NHCH₃, or —CH₂CH₂NH₂.
8. The compound of embodiment 6 or 7 wherein R⁶ is CF₃.
9. The compound of any one of embodiments 6-8 wherein R² and R³ are H.
10. The compound of any one of embodiment 6-9 wherein R⁴ and R⁵ are H.
11. The compound of embodiment 1 wherein formula II is represented by formula IV or V:

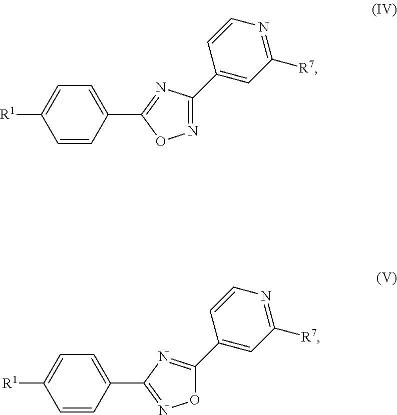

or
a pharmaceutically acceptable salt thereof;
wherein,
   R¹ is —CH₂NH₂, —CH₂NHCH₃, or —CH₂CH₂NH₂; and
   R⁷ is Ar or OAr, wherein Ar is:

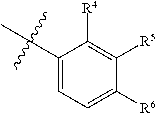

wherein,
   R⁴ is H, CH₂(halo), or NO₂;
   R⁵ is H or halo; and
R⁶ is H, halo, CF₃, —C(═O)CH₃, or —C≡CH.
12. The compound of embodiment 11 wherein R¹ is —CH₂NH₂.
13. The compound of embodiment 11 or 12 wherein R¹ is 4-(trifluoromethyl)phenyl or oxy-4-(trifluoromethyl)phenyl.
14. The compound of embodiment 1 wherein the compound is:

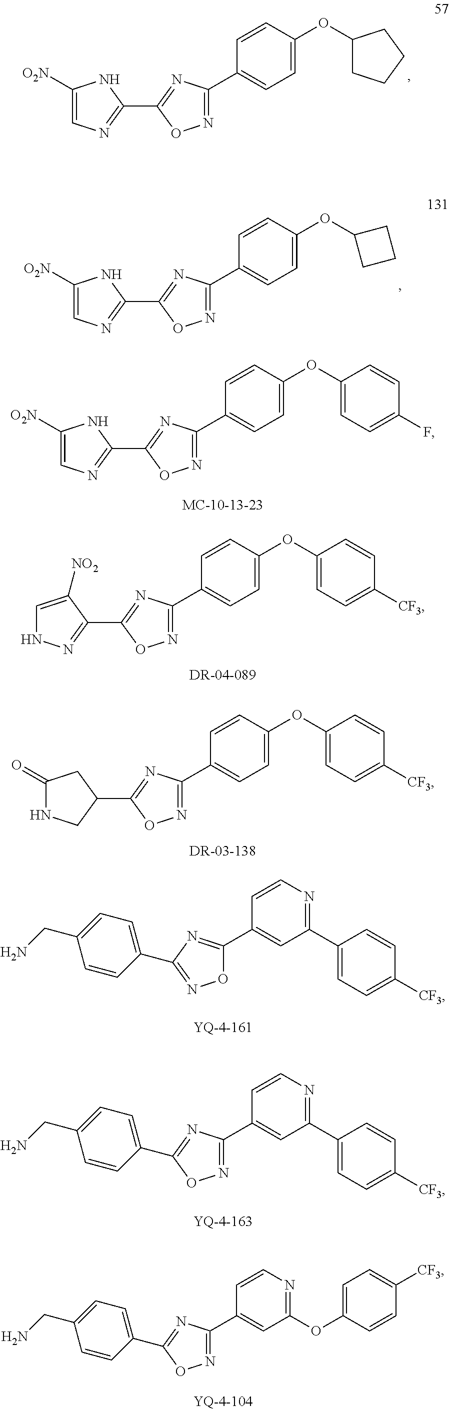

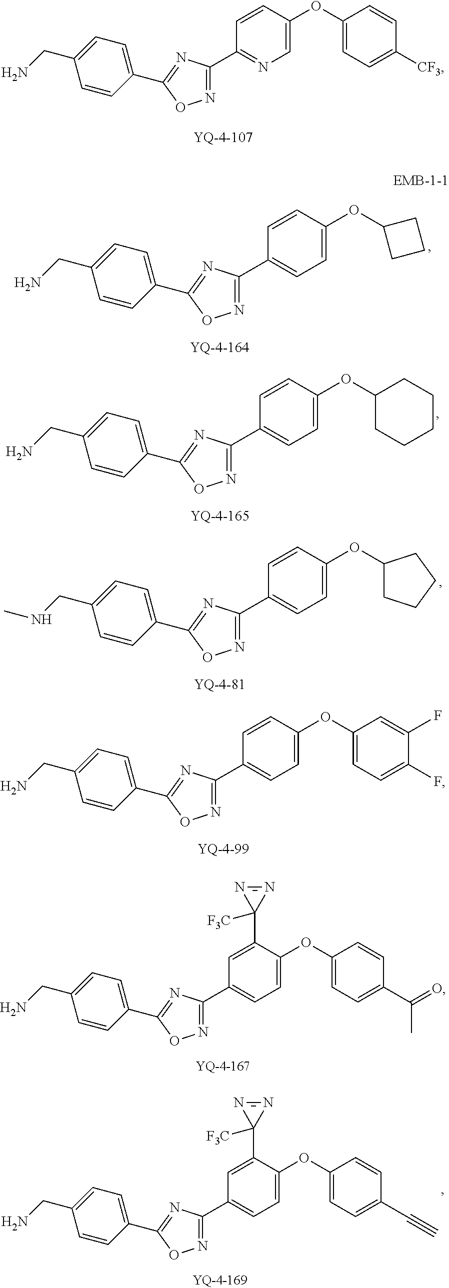

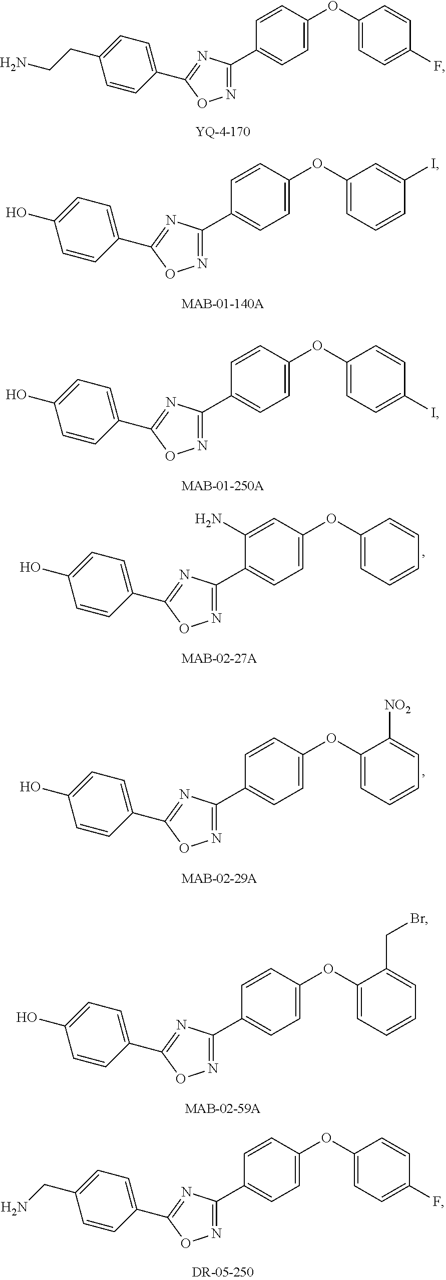

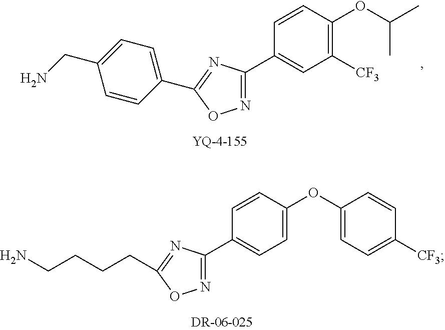 [SMILES: CC(C)Oc1ccc(-c2noc(-c3ccc(CN)cc3)n2)cc1C(F)(F)F ; NCCCCc1nc(-c2ccc(Oc3ccc(C(F)(F)F)cc3)cc2)no1]

or
   a pharmaceutically acceptable salt thereof.
15. A method for treating a Clostridioides difficile infection (CDI), and/or preventing the recurrence of CDI comprising administering to a subject having a CDI and/or at risk of CDI recurrence, a therapeutically effective dose of a compound of any one of embodiments 1-14 or a compound otherwise described herein, wherein the compound inhibits growth of a Clostridioides difficile vegetative cell and/or germination of a Clostridioides difficile spore that is present in the CDI and the subject is thereby treated.
16. The method of embodiment 15 wherein the Clostridioides difficile spore is in a vegetative state.
17. The method of embodiment 15 or 16 wherein the compound selectively inhibits germination of a Clostridioides difficile spore by damaging the cell wall of the spore.
18. The method of any one of embodiments 15-17 wherein the compound is inactive at inhibiting microbiome gut bacteria (e.g., gut flora that grow in the gastrointestinal tract that are beneficial to human health) in the subject.
19. The method of any one of embodiments 15-18 wherein the compound is inactive at inhibiting Gram-negative bacteria and/or Gram-positive bacteria, in some embodiments-including mature Clostridioides difficile, and in other embodiments, excluding mature Clostridioides difficile.
20. The method of any one of embodiments 15-19 wherein the compound is 3-(4-(cyclopentyloxy)phenyl)-5-(5-nitro-1H-imidazol-2-yl)-1,2,4-oxadiazole (57).

Further embodiments of the technology disclosed herein include:
15A. A method for treating a Clostridioides difficile infection (CDI) comprising administering to a subject having a CDI a therapeutically effective dose of a compound of any one of embodiments 1-14 or a compound otherwise described herein, wherein the compound inhibits growth of a Clostridioides difficile vegetative cell or germination of a Clostridioides difficile spore that is present in the CDI and the subject is thereby treated.
15B. A method for treating a Clostridioides difficile infection (CDI) and preventing CDI recurrence comprising administering to a subject having a CDI a therapeutically effective dose of a compound of any one of embodiments 1-14 or a compound otherwise described herein, wherein the compound inhibits both growth of a Clostridioides difficile vegetative cell and germination of a Clostridioides difficile spore that is present in the CDI and the subject is thereby treated.
15C. A method for treating a Clostridioides difficile infection (CDI) and preventing CDI recurrence comprising administering to a subject having a CDI a therapeutically effective dose of a compound of any one of embodiments 1-14 or a compound otherwise described herein, wherein the compound inhibits both growth of a Clostridioides difficile vegetative cell and germination of a Clostridioides difficile spore that is present in the CDI and the subject is thereby treated.
15C.1. The method of embodiment 15C wherein the compound inhibits growth of a Clostridioides difficile vegetative cell at higher doses and germination of a Clostridioides difficile spore at lower doses.
15D A method for treating a Clostridioides difficile infection (CDI) and preventing recurrent Clostridioides difficile infection (CDI) comprising administering to a subject at risk of recurrent CDI a therapeutically effective dose of a compound of any one of embodiments 1-14, or a compound otherwise described herein, in combination with and/or following an antibiotic that treats CDI, wherein the compound inhibits germination of a Clostridioides difficile spore and the subject is thereby treated. Antibiotics that treat CDI and that can be used in combination therapy with a compound described herein include, but are not limited to, vancomycin, metronidazole, fidaxomicin, amikacin, streptomycin, doxycycline, erythromycin, gentamicin, isoniazid, rifampin, ethambutol, clindamycin, and clindamycin phosphate.
15E. A method for preventing an initial Clostridioides difficile infection (CDI) or recurrent CDI comprising administering to a subject at risk of initial or recurrent CDI a therapeutically effective dose of a compound of any one of embodiments 1-14 or a compound otherwise described herein, wherein the compound inhibits germination of a Clostridioides difficile spore and the subject is thereby treated.

### Results and Discussion.

Synthesis. A three-step synthetic route was devised for the preparation of the 1,2,4-oxadiazole antibacterials (Scheme 1). Nucleophilic substitution or Ullmann coupling on the benzonitrile species 3 produced the corresponding ethers 5, per known methodology. The resulting derivatized benzonitriles (compounds 5) were typically allowed to react with hydroxylamine, yielding the corresponding amidoximes (6) (Scheme 1). The construction of the 1,2,4-oxadiazoles (compounds 8) would utilize the amidoximes in conjunction with carboxylic acids 7 by the three methods outlined under step c of Scheme 1. In method A, the carboxylic acid was pretreated with 1, l′-carbonyldiimidazole (CDI) and then left to react with amidoxime at room temperature to form an acyclic intermediate, before cyclization at 140° C. in DMF. For method B, carboxylic acids 7 were coupled with amidoxime in the presence of dicyclohexylcarbodiimide (DCC), prior to cyclization in refluxing 1,4-dioxane. Alternatively, in method C, a carboxylic acid is refluxed with thionyl chloride to form the corresponding acyl chloride, which then was allowed to react with amidoxime in refluxing pyridine.

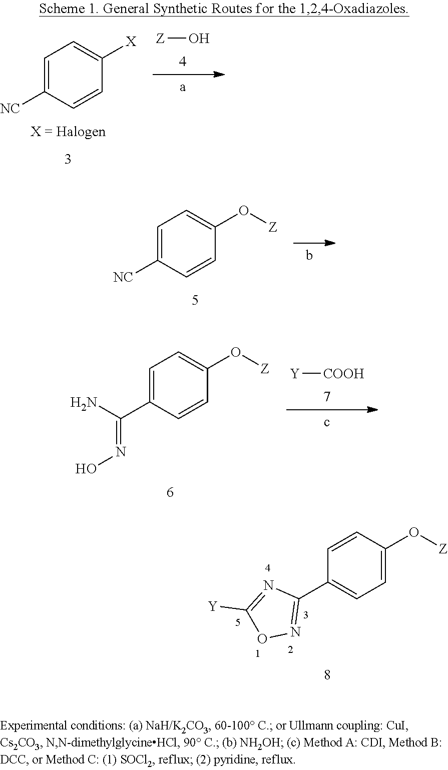

Other useful synthetic techniques and assays are described by U.S. Pat. No. 11,168,062 (Chang et al.), which is incorporated herein by reference.

Structure-Activity Relationship for 1,2,4-Oxadiazoles. All 133 analogs, spanning three SAR groups, were tested against C. difficile strain ATCC 43255, a clinical isolate from an abdominal wound that produces toxins A and B, but not the binary toxin CDT. MIC values were assessed after 48-h incubation (Chart 1-3).

Chart 1. Antibacterial activities of the 1,2,4-oxadiazole derivatives for SAR1. The MIC values (μg/mL) were determined with strain C. difficile ATCC 43255. The MICs for inactive (MIC≥8 μg/mL) and active compounds (MIC≤4 μg/mL) are shown.

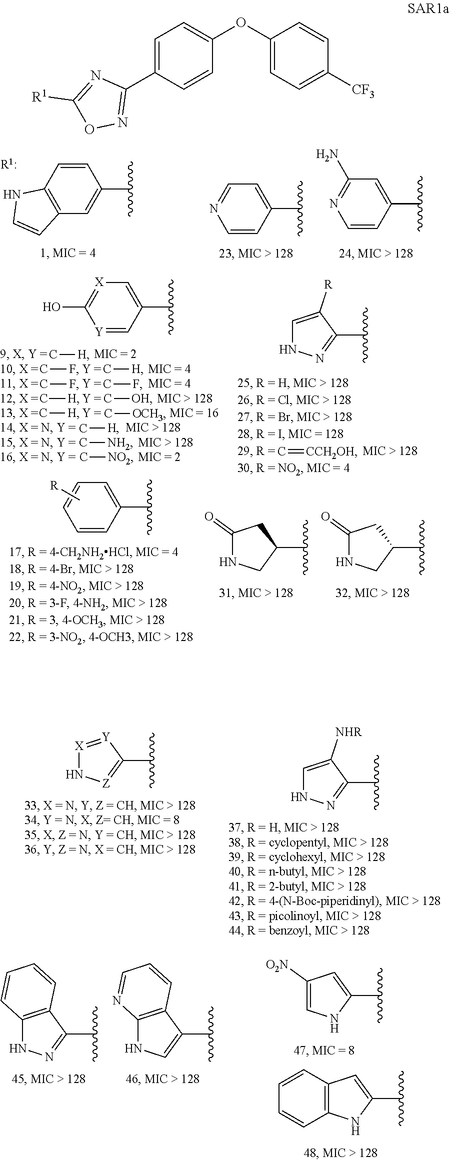

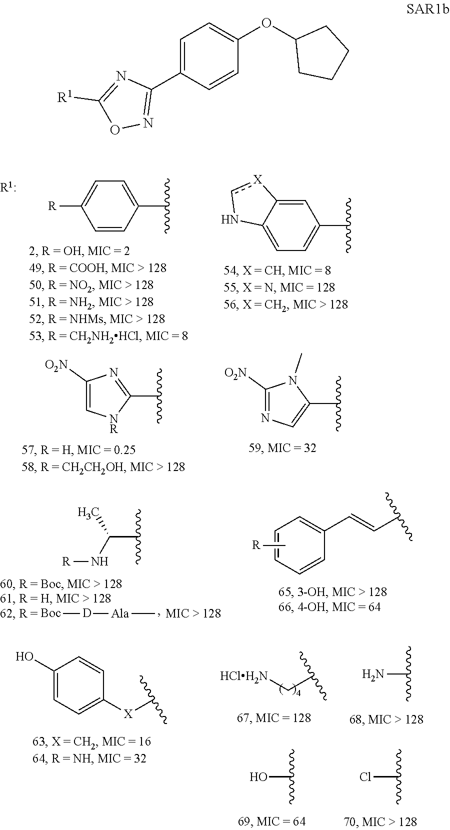

The SAR1 study explored the nature of the 5-substituent on the oxadiazole ring (R¹, Chart 1) while keeping the 3-substituents fixed to the trifluoromethyl diphenyl ether in SAR1a and the cyclopentyl phenyl ether in SAR1b. In SAR1a, with the 4-hydroxylphenyl substitution as R¹ group, compound 9 showed better antibacterial activity (MIC=2 μg/mL) than 1 with an indole group. Upon this discovery, compounds 10 to 16 were explored in addition to the 4-hydroxylphenyl group. Interestingly, only compounds with electro-withdrawing substituents on the phenol substructure retained good activity. For example, fluorinated analogs 10 and 11 along with nitro-substituted compound 16, all exhibited MICs in the 2-4 μg/mL range. Further introduction of electron-donating groups such as —OH or —OMe (12 and 13, respectively) abrogated or diminished activity against C. difficile (MIC=128 and 16 μg/mL, respectively). Replacing the phenol with the 2-pyridone substructure-tautomeric lactam-lactim-resulted in loss of activity (14 and 15, MIC>128 μg/mL). We next changed the hydroxyl group on the phenyl ring for various substituents (17-24). The 4-aminomethylphenyl analog 17 had the same MIC (4 μg/mL) as the indole 1. All other attempts at analogs at this site led to loss of activity (MIC>128 μg/mL). A number of analogs were prepared in which we introduced five-membered heterocycles at the C-5 of the oxadiazole (25-48). Surprisingly, only oxadiazole with a 4-nitro-1H-pyrazole moiety (30) retained activity with MIC of 4 μg/mL; the imidazole bearing oxadiazole 34 had decreased activity (MIC of 8 μg/mL). Other analogs, including the ones that exhibited activity against MRSA (26-29, Table 3 in Example 3), conferred no antibacterial activity against C. difficile.

In SAR1b, twenty-three analogs with cyclopentyl phenyl ether (2, 49-70) were prepared and evaluated. Compound 2 with p-phenol group showed equivalent antibacterial activity to that of compound 9 (MIC=2 μg/mL). Compounds 53 and 54 had comparable antibacterial effects (2× decrease, MIC=8 μg/mL) as to their counterparts in SAR1a (17 and 1). Compared to 2, compounds with phenol bioisosteres (49-52, 55, 56) lost their activity against C. difficile (MIC>128 μg/mL). Notably, the aniline-containing analog 51 exhibited an MIC of 2 μg/mL after 24-h incubation with C. difficile vegetative cells, yet lost activity (>128 μg/mL) after 48-h incubation (Table 3). We attributed this finding to persister cells, which emerged on the second day of incubation. The 4-nitro-1H-imidazole-containing compound 57 showed potent activity at 0.25 μg/mL. Regioselective hydroxyethylation of the imidazole ring nitrogen of compound 57 led to 58, which lost activity. Another nitroimidazole analog 59 with 1-methyl-2-nitro-1H-imidazole moiety showed decreased activity of 32 μg/mL. A few analogs with acyclic moieties (60-67) or those with removal of 5-substituents (68-70) were also prepared, but none conferred anti-C. difficile activity (MIC=64 or >128 μg/mL).

The SAR2 study focused on the structural exploration of the 3-substituent on the oxadiazole ring (R², Chart 2) with the four priority substructures (phenol, nitropyrazole, indole and nitroimidazole; highlighted in blue, SAR2a-c) that emerged in SAR1 of the 1,2,4-oxadiazole ring. In SAR2a, phenyl ethers that contain aromatic (71-82) or aliphatic (83-99) groups were first assessed as R² substituents. Diphenyl ethers as R² substituents were beneficial for anti-C. difficile activity, with compound 71 retaining activity (MIC=2 μg/mL). Further substitutions on the terminal phenyl ring like 2-NO₂ (72) and 3-I (76) were well tolerated with an observed MIC of 4 μg/mL, while substitutions like 2-CH₂Br (73), 4-OH (74), 4-CN (75) and 4-I (77) led to loss of activity with MICs of 8-128 μg/mL. Modifications on the phenyl ring that connected directly to the 3-position of the 1,2,4-oxadiazole were also explored (78-82). The lack of activity of these compounds (MIC>128 μg/mL) indicated that insertion of heteroatoms and substitution of NH₂, NO₂ or Br were detrimental to the activity against C. difficile.

We next evaluated additional aliphatic rings. The aliphatic groups (83-99) were selected to diversify steric, electronic, polar, and hydrogen-bonding properties of the functionalities. Compound 99 with a six-membered aliphatic ring retained activity (MIC=2 μg/mL), while all of the other analogs remained inactive. Unfortunately, compounds 85, 87, and 94 had the persister issues with >8-fold activity decrease at 48-h compared to the 24-h results (Table 3). Given the activity of compounds 48 (containing a cyclopentane ring) and 99 (with a cyclohexane ring), a saturated five- or six-membered ring appears to be favored for anti-C. difficile activity.

For compounds 100-101, attempts to replace the diphenyl ether subunit with a N-phenylpiperazinyl moiety led to loss of activity. The last series in this study dealt with replacement of the diphenyl ether with a more rigid dibenzofuran ring (102-105) or modifying the phenyl moiety (106-114), all of which showed reduced activity (MIC≥8 μg/mL).

For the remaining examples of SAR2, nitro-pyrazole, indole and nitroimidazole substituents were incorporated at the C-5 position of the central 1,2,4-oxadiazole ring system. In SAR2b, nitro-pyrazole bearing analogs 115, 117 and 118 generally showed better antibacterial effects against C. difficile than their counterparts in SAR2a (85, 77 and 102). In SAR2c, all attempts (120-129) led to loss of activity. In SAR2d, the nitroimidazole-bearing analog 131 maintained potent MIC (0.25 μg/mL) on par with compound 57. However, attempts to remove the ether (130) or insert (132-134) nitrogen atoms in the distal cyclopentyl (or cyclohexyl) ring of compound 57 led to loss of the activity (MIC>128 μg/mL).

The SAR3 study focused on modifying the oxadiazole ring. Derivatives with isoxazole (135), triazole (136), reversed 1,2,4-oxadiazole (137), diformylhydrazine (138) and 1,3,4-oxadiazole (139) cores were evaluated. Only compounds with the reversed 1,2,4-oxadiazole (137) retained activity (MIC=4 μg/mL), which was slightly poorer than 2 with the original substitution pattern on the 1,2,4-oxadiazole core. Much to our surprise, the derivative with the 1,3,4-oxadiazole ring, along with the ones with isoxazole and triazole rings, confer no activity (MIC>128 μg/mL).

Chart 2. Antibacterial activities of the 1,2,4-oxadiazole derivatives for SAR2. The MICs (μg/mL) were determined with C. difficile ATCC 43255. The MICs for inactive (MIC≥8 μg/mL) and active compounds (MIC≤4 μg/mL) are shown.

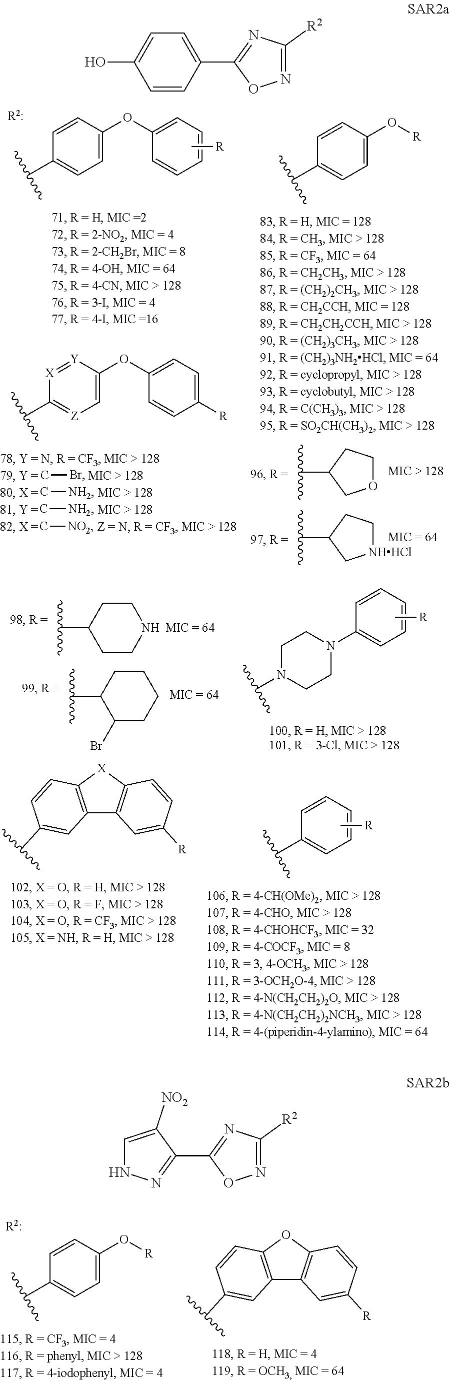

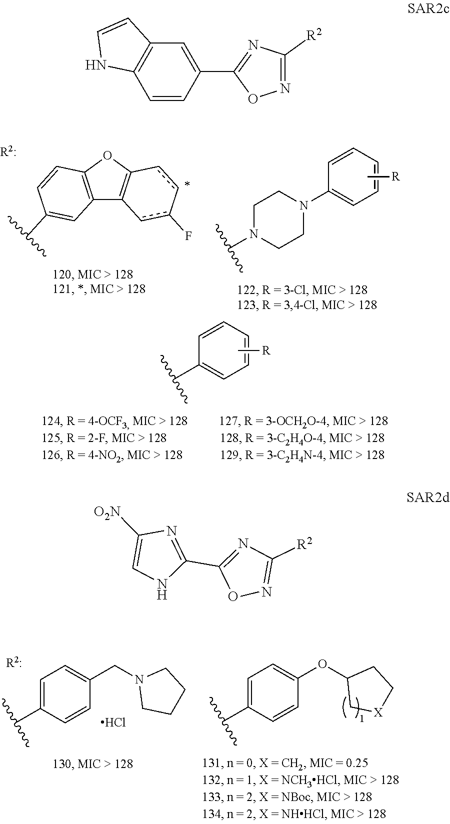

Chart 3. Antibacterial activities of the 1,2,4-oxadiazole derivatives for SAR3. The MICs (μg/mL) were determined with C. difficile ATCC 43255. The MICs for inactive (MIC≥8 μg/mL) and active compounds (MIC≤4 μg/mL) are shown.

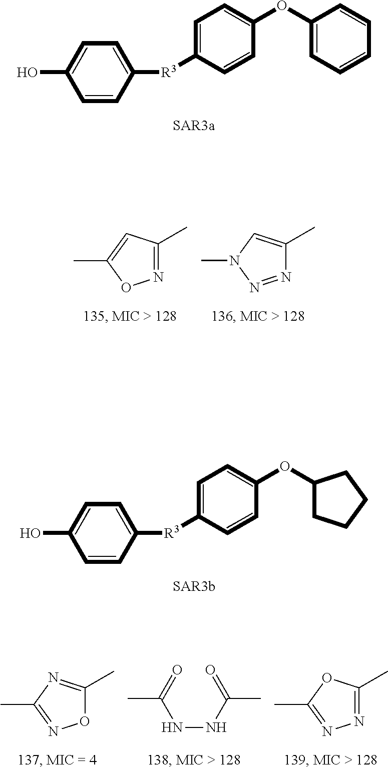

In Vitro Toxicity. Overall, 18 analogs exhibited MIC≤4 μg/mL against C. difficile strain ATCC 43255. These compounds were further evaluated in in vitro toxicity using the lactate dehydrogenase (LDH) assay with THP-1 (human monocyte) cells (Table 1). LDH is present in the cytosol; damage to the plasma membrane results in LDH release into the cell culture medium. The extracellular LDH is quantified by conversion of lactate to pyruvate, which reduces NAD⁺ to NADH; the latter is used to reduce a tetrazolium salt to a red formazan that is proportional to the amount of LDH released and it is measured at 490 nm. Four compounds had toxicity IC₅₀ values above 50 μg/mL (1, 57, 117, and 131). Of these two oxadiazoles had acceptable selectivity (IC₅₀/MIC)>50 (57 and 131). We evaluated compounds 57 and 131 with a second cell viability and proliferation XTT assay using HepG2 (liver) cells. This assay is based on the reduction of XTT, a yellow tetrazolium salt, to an orange formazan carried out by mitochondrial enzymes of healthy cells. The higher absorbance indicates higher mitochondrial enzyme activity and higher cell viability. The XTT IC₅₀ values were 53.4±4.5 μg/mL for 57 (IC₅₀/MIC ratio of 214) and 41.9±4.5 μg/mL (IC₅₀/MIC ratio of 168) for 131. We focused on compound 57.

TABLE 1
In vitro toxicity of selected active oxadiazoles.

| | MIC_{C. difficile} | LDH (IC₅₀) | IC₅₀/MIC |
|---|---|---|---|
| Compound | (μg · mL⁻¹) | (μg · mL⁻¹) | (×) |
| 1 | 4 | 69 ± 6 | 17 |
| 2 | 2 | 18 ± 1 | 9 |
| 9 | 2 | 17 ± 1 | 9 |
| 10 | 4 | 5.2 ± 0.02 | 1 |
| 11 | 4 | 18 ± 2 | 4 |
| 16 | 2 | 37 ± 11 | 19 |
| 17 | 4 | 4.4 ± 0.7 | 1 |
| 30 | 4 | 21 ± 5 | 5 |
| 57 | 0.25 | >128 | >512 |
| 71 | 2 | 17 ± 1 | 8 |
| 72 | 4 | 16 ± 1 | 3 |
| 76 | 4 | 17 ± 0.2 | 4 |
| 99 | 2 | 8.4 ± 0.3 | 4 |
| 115 | 4 | 25 ± 4 | 6 |
| 117 | 4 | 100 ± 9 | 25 |
| 118 | 4 | 9.8 ± 3.3 | 2 |
| 131 | 0.25 | >128 | >512 |
| 137 | 4 | 8.5 ± 0.2 | 2 |

Bactericidal Activity. An MBC/MIC≤4 is an indication of bactericidal activity. We evaluated whether oxadiazole 57 is bactericidal or bacteriostatic by determination of the minimal-bactericidal concentration (MBC) against C. difficile ATCC 43255 strain. The MBC of 57 is 0.5 (2-fold higher than MIC), indicating that it is bactericidal. Likewise, MTZ (MBC/MIC=1) and FDX (MBC/MIC=1) are bactericidal. In contrast, VAN is bacteriostatic (MBC/MIC=32). This is in agreement with the earlier reported for MTZ, FDX, and VAN.

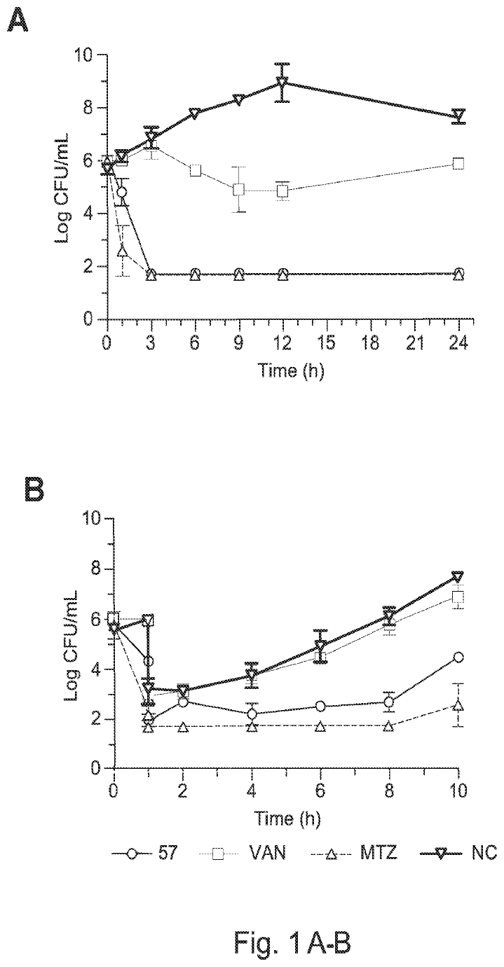

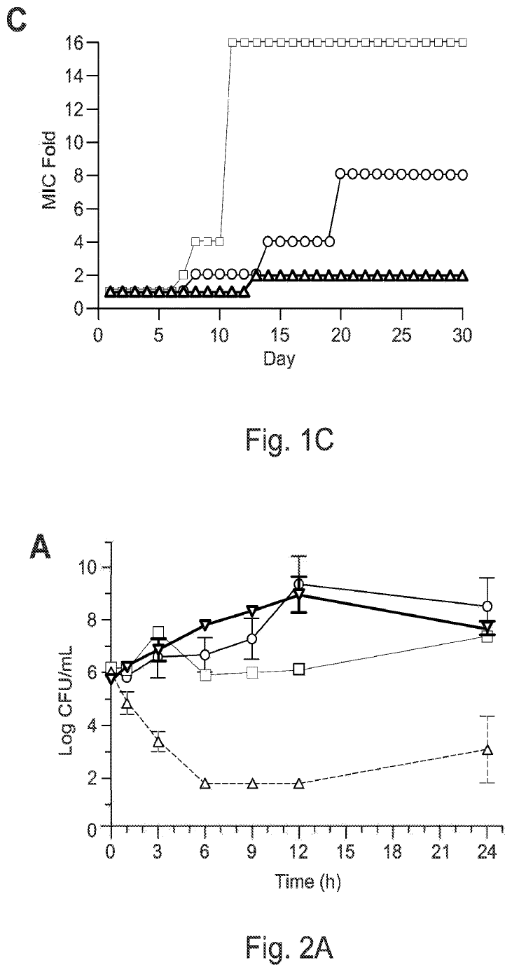

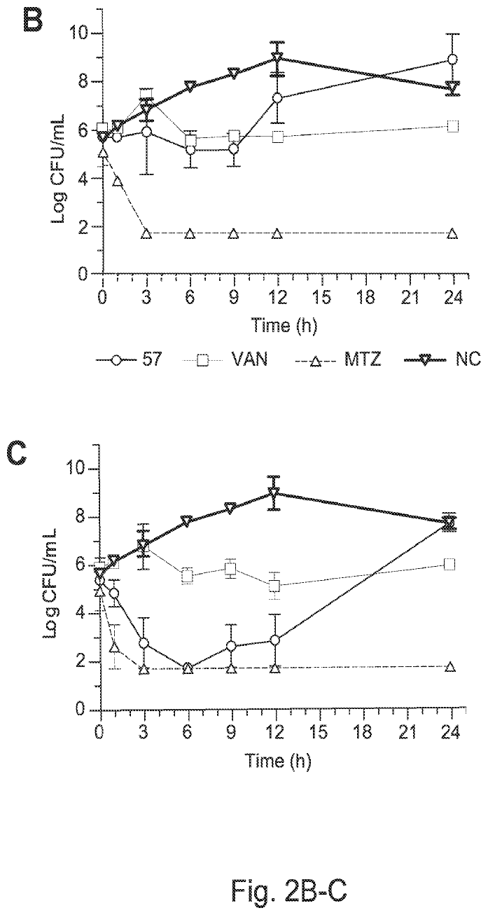

Time-kill. The effect of compound 57 on C. difficile ATCC 43255 growth as a function of time was evaluated at 1×, 2×, 4×, and 8× MIC (FIG. 1A and FIG. 2). Four-log₁₀ reduction in bacterial growth was observed at all concentrations, consistent with bactericidal activity, as defined by reduction of ≥3 log 10 or ≥99.9%. The time to achieve 4-log₁₀ reduction in bacterial load decreased as the concentration increased.

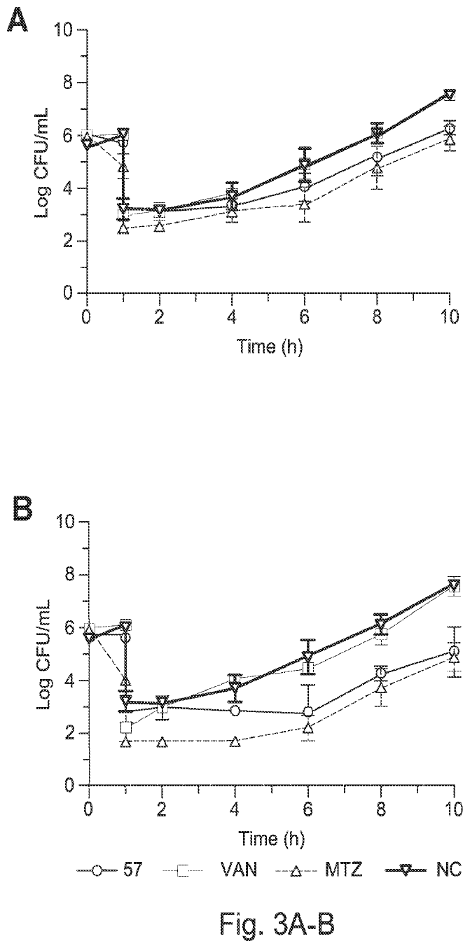

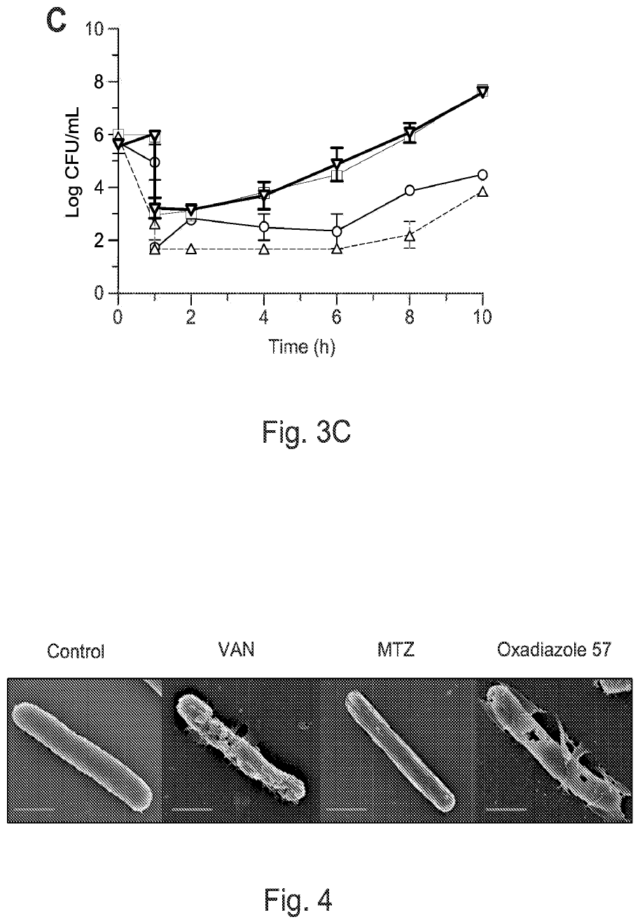

Post-antibiotic Effect (PAE). The PAE is the time of bacterial growth suppression after removal of the antibiotic. The PAE was investigated for compound 57 using ATCC43255 after 1000-fold dilution of the oxadiazole following a 1-h exposure at 1×, 2×, 4×, and 8× MIC, using VAN and MTZ as positive controls (FIG. 1B and FIG. 3). The PAE was 3 h at 1× MIC, increasing to 5 h at 2× MIC, 7 h at 4× MIC, and 7 h at 8× MIC. VAN had a short PAE of 1 h at 1×, 2×, and 8× MIC, and 3 h at 4× MIC.

Emergence of Resistance. Serial passage of C. difficile ATCC 43255 was conducted to evaluate emergence of resistance (FIG. 1C). The MIC of 57 increased 2-fold after 8 passages, 4-fold after 14 passages, and 8-fold after 20 passages. The MIC of VAN increased 2-fold after 7 passages, 4-fold after 8 passages, 8-fold after 11 passages, and 16-fold after 15 passages. In contrast, the MIC of MTZ increased 2-fold after 13 passages and did not increase further after 30 passages.

Mode of Action. The mechanism of action of oxadiazole 57 was investigated by scanning electron microscopy (SEM) using C. difficile ATCC 43255 (FIG. 4). Vegetative cells treated with 57 showed damage to the cell wall. The same effect was observed in cells treated with VAN, a known cell-wall-synthesis inhibitor. No obvious effect was seen in MTZ-treated cells.

Activity against Additional C. difficile Strains. Additional Gram-positive and Gram-negative Bacteria, and Common Gut Bacteria. We evaluated the activity of oxadiazole 57 with 101 C. difficile strains from our collection. MIC values ranged from 0.125 to 2 μg/mL, with MIC₅₀ of 0.5 μg/mL and MIC₉₀ of 1 μg/mL (Table 2 and Table 4 in Example 4), the same as MTZ and better than VAN. The corresponding values for the clinically-used antibiotics as comparators are given in Table 2. The MIC₅₀ and MIC₉₀ values for oxadiazole 57 are 4-fold lower than those for oxadiazole 2 (Table 2).

As oxadiazoles 1 and 2 exhibit antibacterial activity against Gram-positive bacteria, we evaluated the spectrum of activity of oxadiazole 57 against an extended panel of Gram-positive bacteria (Table 2). Oxadiazole 57 did not exhibit activity against the additional Gram-positive bacteria in Table 2 (MIC values ranging from 32 to >128 μg/mL). Like oxadiazole 57, MTZ did not display activity against other Gram-positive strains with MICs ranging from >16 to >128 μg/mL. The activity of FDX against the panel of Gram-positive bacteria was modest, with MICs ranging from 2 to >16 μg/mL. On the other hand, VAN has activity against Gram-positive bacteria. Oxadiazole 57 showed no activity against common gut bacteria (MICs 32 to >128 μg/mL), in contrast to oxadiazoles 1 and 2 (MICs 0.5 to >128 μg/mL). Likewise, VAN, MTZ, and FDX showed activity against some common gut bacteria, with MIC values of 0.25 to >32 μg/mL for VAN, 1 to >32 for MTZ, and ≤0.01 to >32 μg/mL for FDX. Oxadiazole 57, as well as oxadiazoles 1 and 2, VAN, MTZ, and FDX, showed no activity against important Gram-negative organisms used in Table 2.

The selectivity of oxadiazole 57 towards C. difficile is a unique feature of this compound. The normal gut microflora prevents colonization of C. difficile. In humans, patients with recurrent CDI have decreased fecal microbiome diversity compared to those with non-recurrent CDI. As the main cause of recurrent CDI is gut microbiota dysbiosis, the narrow-spectrum activity of oxadiazole 57 has the potential to stop recurrence of CDI.

TABLE 2
MIC values for oxadiazoles 1, 2 and 57, VAN, MTZ, and FDX against C. difficile
strains, extended Gram-positive, common gut bacteria, and Gram-negative bacteria.

| | | | | | | |
|---|---|---|---|---|---|---|
| Organism | 1 ^{a} | 2 ^{a} | 57 | VAN ^{b} | MTZ ^{b} | FDX ^{b} |

C. difficile Strains

| | | | | | | |
|---|---|---|---|---|---|---|
| C. difficile ATCC43255 ^{c} | 2 | 2 | 0.25 | 0.5 | 0.25 | 0.06 |
| C. difficile BAA-1870 ^{d} | 4 | 2 | 0.125 | 2 | 0.5 | 0.25 |
| C. difficile BAA-1803 ^{e} | 2 | 2 | 0.125 | 2 | 0.5 | 1 |
| C. difficile NR49310 ^{f} | 2 | 4 | 0.25 | 0.5 | 0.125 | ≤0.01 |
| C. difficile NR49294 ^{g} | 1 | 2 | 0.5 | 0.25 | 0.25 | 0.01 |
| C. difficile NR49305 ^{h} | 4 | 2 | 0.125 | 0.5 | 0.25 | 0.01 |
| C. difficile NR49318 ⁱ | 2 | 4 | 0.5 | 0.25 | 0.25 | 0.125 |
| C. difficile NR49292 ^{j} | 2 | 2 | 0.125 | 0.25 | 0.25 | 0.5 |
| C. difficile NR49302 ^{k} | 2 | 2 | 0.25 | 0.5 | 0.125 | 0.03 |
| C. difficile 26675 ^{l} | 4 | 4 | 2 | 1 | 4 | 4 |
| C. difficile 23828 ^{m} | 4 | 4 | 1 | 2 | 0.5 | >8 |
| C. difficile 24531 ⁿ | 4 | 4 | 0.125 | 4 | 0.5 | 0.01 |
| IMIC₅₀ (101 strains) | 4 | 2 | 0.5 | 0.5 | 0.5 | 0.06 |
| MIC₉₀ (101 strains) | 8 | 4 | 1 | 2 | 1 | 0.25 |

Extended Gram-positive Bacteria

| | | | | | | |
|---|---|---|---|---|---|---|
| S. aureus ATCC 29213 ^{o} | 4 | 2 | 64 | 1 | >256 | 16 |
| S. aureus ATCC 27660 ^{p} | 4 | 2-4 | 32 | 2 | >256 | 16 |
| S. aureus NRS100 (COL) ^{p} | 2 | 1 | 64 | 2 | >256 | 16 |
| S. aureus NRS119 ^{q} | 2 | 1-2 | 32 | 1 | >256 | 4 |
| S. aureus NRS120 ^{q} | 2 | 2 | 64 | 1 | >256 | 8 |
| S. aureus VRS1 ^{r} | 2 | 2 | 64 | 64 | >256 | 64 |
| S. aureus VRS2 ^{s} | 2 | 2 | 32 | 32 | >256 | 8 |
| S. epidermidis ATCC 35547 | 2 | 2 | 64 | 4 | >128 | 4 |
| S. haemolyticus ATCC 29970 | 8 | 2 | 32 | 4 | >256 | 16 |
| S. oralis ATCC 9811 | 128 | >128 | 64 | 1 | >128 | 32 |
| S. pyogenes ATCC 49399 | 64 | >128 | >128 | 0.5 | >128 | 8 |
| B. cereus ATCC 13061 | 16 | 2 | 64 | 1 | >256 | 16 |
| B. licheniformis ATCC 12759 | 8 | 4 | 64 | 0.5 | >256 | 8 |
| E. faecalis ATCC 29212 ^{o} | 8 | 4 | 64 | 2 | >256 | 16 |
| E. faecalis 201 (Van S) ^{t} | 4 | 4 | 64 | 1 | >256 | 16 |
| E. faecalis 99 (Van R) ^{u} | 4 | 4 | 128 | 128 | >256 | 16 |
| E. faecium 119-39A (Van S) ^{t} | 1 | 2 | 128 | 0.5 | >16 | >16 |
| E. faecium 106 (Van R) ^{u} | 2 | 2 | 128 | 128 | >16 | >16 |
| E. faecium NCTC 7171 | 2 | 4 | 64 | 0.5 | >128 | 2 |

Common Gut Bacteria

| | | | | | | |
|---|---|---|---|---|---|---|
| Bacteroides fragilis HM-709 ^{v} | 4 | 2 | 128 | 16 | 1 | >32 |
| Bifidobacterium longum HM-846 ^{w} | 0.25 | 1 | 32 | 0.25 | 0.05 | <0.01 |
| Corynebacterium spp. HM-784 ^{x} | 2 | 0.5 | >128 | 0.5 | >32 | <0.06 |
| Fusobacterium nucleatum HM-992 ^{y} | 1 | 2 | 64 | 0.25 | 2 | <0.06 |
| Lactobacillus reuteri HM-102 ^{z} | >128 | >128 | >128 | >32 | >32 | >32 |
| Lactobacillus gasseri HM-644 ^{aa} | >128 | >128 | >128 | 1 | >32 | 2 |
| Veillonella sp. HM-49 ^{bb} | >128 | >128 | 32 | >32 | 2 | 8 |
| Eubacterium sp. HM-178 ^{cc} | 2 | 4 | 64 | 2 | 1 | 16 |

Gram-negative Bacteria

| | | | | | | |
|---|---|---|---|---|---|---|
| K. pneumoniae ATCC700603 | >128 | >128 | >128 | >128 | >128 | >128 |
| A. baumanii ATCC17961 | >128 | >128 | >128 | >128 | >128 | >128 |
| P. aeruginosa ATCC27853 | >128 | >128 | >128 | >128 | >128 | >128 |
| E. aerogenes ATCC35029 | >128 | >128 | >128 | >128 | >128 | >128 |
| E. coli ATCC25922 | >128 | >128 | >128 | >128 | >128 | >128 |

^{a} Data from Janardhanan et al., reproduced for the sake of comparison (Proc. Natl. Acad. Sci. USA. 2023, 120, e2304110120).
^{b} MICs for VAN, MTZ, and FDX in C. difficile strains and common gut bacteria from Speri et al.; reproduced for the sake of comparison (ACS Infect. Dis. 2020, 6, 2362).
^{c} isolated from abdominal wound, TcdA+, TcdB+.
^{d} NAP1, BI 8, ribotype 27, toxinotype IIIb, TcdA+, TcdB+, CDT+ (binary toxin).
^{e} clinical isolate, NAP1, toxinotype IIIc, ribotype 027, tcdA+, tcdB+, cdtB+.
^{f} isolated from human feces, NAP7, ribotype 078, TcdA+, TcdB+, TcdC+ (Δ39), CDT+.
^{g} isolated from human feces, NAP4, ribotype 014, TcdA+, TcdB+, TcdC+, CDT−, most prevalent after NAP1/027.
^{h} isolated from human feces, NAP6, ribotype 002, TcdA+, TcdB+, TcdC+, CDT−, community associated epidemic strain.
ⁱ isolated from human feces, NAP11, ribotype 106, TcdA+, TcdB+, TcdC+, CDT−, predominant epidemic strain in a children's hospital in Chicago, increased risk of relapses.
^{j} isolated from human feces, NAP2, ribotype 001_072, TcdA+, TcdB+, TcdC+, CDT−, epidemic in US during 1990s and still common.
^{k} isolated from human feces, NAP4, ribotype 020, TcdA+, TcdB+, TcdC+, CDT−, among top 7 isolates in 2011-2012.
^{l} clinical isolate resistant to MTZ.
^{m} clinical isolate resistant to FDX.
ⁿ clinical isolate resistant to VAN.
^{o} a quality control strain to monitor accuracy of MIC testing.
^{p} mecA positive, resistant to methicillin, oxacillin, and tetracycline; susceptible to vancomycin and linezolid.
^{q} mecA positive, resistant to ciprofloxacin, gentamicin, oxacillin, penicillin, and linezolid.
^{r} vancomycin-resistant MRSA (vanA) clinical isolate from Michigan.
^{s} vancomycin-resistant MRSA (vanA) clinical isolate from Pennsylvania.
^{t} vancomycin-susceptible clinical isolate.
^{u} vancomycin-resistant clinical isolate.
^{v} Strain HM-709, Gram-negative, anaerobic bacterium that is commensal and critical to host immunity; a minor component of the human gut microflora (<1%).
^{w} Strain HM-846, anaerobic, Gram-positive bacterium commonly found in the normal human intestinal microflora isolated from human feces, nonsporulating.
^{x} Strain HM-784, Gram-positive, aerobic or facultatively anaerobic bacterium that occurs in the mucosa and normal skin flora of humans and animals.
^{y} Strain HM-992, anaerobic, nonsporulating, Gram-negative bacterium commonly found in the gastrointestinal tract.
^{z} Strain HM-102, Gram-positive, anaerobic bacteria commonly found in the normal human gastrointestinal tract, commonly used as a probiotic to maintain the balance of gut microbial flora.
^{aa} Strain HM-644, Gram-positive, facultative, anaerobe bacterium commonly found in the normal human gastrointestinal tract, commonly used in yogurt production as a probiotic to suppress Helicobacter pylori infections.
^{bb} Gram negative, nonsporulating bacterium commonly found in the intestinal tract of humans and animals.
^{cc} Strain HM-178, anaerobic, nonsporulating, Gram-positive bacterium commonly found in the gastrointestinal flora of humans and animals.

Conclusion. The discovery of the oxadiazoles active against MRSA, led us to investigate the activity of these compounds against C. difficile, a Gram-positive anaerobic bacterium that is responsible for more deaths in the United States than all four remaining urgent threats combined. We had reported earlier the discovery of oxadiazole 2 with activity against C. difficile (MIC₉₀ of 4 μg/mL). SAR studies of 133 oxadiazole analogs led to the discovery of compound 57, which exhibits selective and potent bactericidal activity (MIC₅₀ (101 strains)=0.5 μg/mL, MIC₉₀ (101 strains)=1 μg/mL). Remarkably, 57 is not active against other Gram-positive organisms, including MRSA. In contrast, its progenitor oxadiazoles 1 and 2 exhibit broad activity against both aerobic, facultative anaerobic, and fully aerobic Gram-positive bacteria. Oxadiazole 57 also has no activity against Gram-negative bacteria and shows poor to no activity against common gut bacteria (MIC ranging from 32 to >128 μg/mL). The ability of 57 to spare gut bacteria is an important property of this compound. The primary cause of recurrent CDI is dysbiosis of gut microbiota. Oxadiazole 57 having poor to no activity against gut pathogens will not disrupt the gut flora, potentially preventing C. difficile colonization.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of enteral administration, e.g., oral administration, sublingual administration, or rectal administration.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms include aqueous solutions, dispersions, or sterile powders comprising the active ingredient, optionally encapsulated in liposomes. The ultimate dosage form should be sterile, fluid and stable under the conditions of manufacture and storage. A liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the compositions can be brought about by agents capable of delaying absorption, for example, aluminum monostearate and/or gelatin.

Various dosage forms can be prepared by incorporating the active compound in the required amount in the appropriate solvent with various other ingredients enumerated above, optionally followed by filter sterilization. Methods of preparation can include vacuum drying and freeze-drying techniques, which yield a powder of the active ingredient plus any additional desired ingredient present in the solution.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be administered orally or sprayed into the mouth using a pump-type or aerosol sprayer. Thickeners such as synthetic polymers, fatty acids, fatty acid salts and esters, fatty alcohols, modified celluloses, or modified mineral materials can also be employed with liquid carriers.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compounds described herein can be effective anti-bacterial agents and have higher potency and/or reduced toxicity as compared to vancomycin. Preferably, compounds of the invention are more potent and less toxic than vancomycin, and/or avoid a potential site of catabolic metabolism encountered with vancomycin and have a different pharmacokinetic profile than vancomycin.

The invention provides therapeutic methods of treating bacterial infections in a mammal, which involve administering to a mammal having bacterial infection an effective amount of a compound or composition described herein. A mammal includes a primate, human, rodent, canine, feline, bovine, ovine, equine, swine, caprine, bovine and the like. Bacterial infections refer to any various type of bacterium that causes debilitating or life-threatening health issues.

The ability of a compound of the invention to treat bacterial infections may be determined by using assays well known to the art. For example, the design of treatment protocols, toxicity evaluation, data analysis, quantification of bacterial cell death, and the biological significance of the use of bacterial screens are known. In addition, ability of a compound to treat bacterial infections may be determined using the protocols as described herein.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

VAN and MTZ were purchased from Sigma Aldrich (St. Louis, MO). All oxadiazole compounds passed the PAINS filter (J. Med. Chem. 2010, 53, 2719). PAINS hits may interfere with biochemical assays by reactivity with nucleophiles, chelation to metals, redox activity, physicochemical, absorption, and fluorescence.

Syntheses. The reagents and solvents were purchased from TCI chemicals (Portland, OR), Sigma-Aldrich (St. Louis, MO), Oakwood Products, Inc. (Estill, SC) or Combi Blocks Inc. (San Diego, CA) and used without further purification. The progress of synthetic transformations was monitored by analytical silica-gel TLC (thin-layer chromatography) plates pre-coated on aluminum foils (200 μm, 60 F₂₅₄, Merck KGaA, Darmstadt, German) under 254 nm UV. Flash column chromatography was performed with CombiFlash® Rf 200i (Teledyne ISCO, Inc., Lincoln, NE) or 60 Å silica gel purchased from Sigma-Aldrich. NMR spectra (¹H, ¹³C, ¹⁹F) were recorded either on a Bruker AVANCE III HD 400 Nanobay (400 MHz for ¹H, 101 MHz for ¹³C and 276 MHz for ¹⁹F, Bruker Biospin AG, Fällanden, France) or a Bruker AVANCE III HD 500 (500 MHz for ¹H and 126 MHz for ¹³C, Bruker Biospin AG, Fällanden, France) at ambient temperature. The residual non-deuterated solvent signals were used as reference (chloroform-d, ¹H δ=7.26 ppm, ¹³C δ=77.2 ppm; DMSO-d₆, ¹H δ=2.50 ppm, ¹³C δ=39.5 ppm; acetone-d₆, ¹H δ=2.05 ppm, ¹³C δ=29.9 ppm; methanol-d₄, ¹H δ=3.31 ppm, ¹³C δ=49.0 ppm). The purity of the compounds was determined by a Dionex Ultimate 3000 HPLC system (Thermo Fisher Scientific Inc., Waltham, MA) with an Acclaim™ RSCL 120 C₁₈ column (0.2 μm, 120 Å, 2.1×100 mm, Thermo Fisher Scientific Inc., Waltham, MA) with UV detection at 254 nm. Elution was at 0.4 mL/min with 90% A/10% B for 1 min, followed by a 7 min linear gradient to 0% A/100% B, then 90% A/10% B for 1.5 min, where A=water and B=acetonitrile. Final compounds showed ≥95% purity. High-resolution mass spectra were recorded using a Bruker micrOTOF/Q2 mass spectrometer (Bruker Daltonics Inc., Fremont, CA) with positive electrospray ionization (ESI+).

Bacterial strains. Antibiotic susceptible C. difficile, Gram-positive, common gut, and Gram-negative strains were obtained from ATCC (Manassas, VA) and BEI Resources (Manassas, VA). VAN-, MTZ-, and FDX-resistant strains were a gift from Dr. Curtis J. Donskey at the Cleveland Veterans Affairs Medical Center, Cleveland, OH, and Drs. Ellie J. C. Goldstein and Diane M. Citron at the R. M. Alden Research Laboratory, Culver City, CA. The strains were cultured and stored according to the suppliers' instructions.

Determination of minimal-inhibitory concentrations. MIC values for C. difficile and the common gut bacteria were determined by the microdilution method using brucella broth (BD Biosciences, Sparks, MD) with hemin and vitamin K or with brain-heart infusion broth (BHIS, BD Biosciences, Sparks, MD). Lactobacillus MRS broth (BD Biosciences, Sparks, MD) was used for culturing Lactobacillus. The concentration of bacteria for these determinations was 5×10⁵ cfu/mL. Two-fold serial dilutions of the compounds were made. Anaerobic bacteria were grown for 48 h at 37° C. in a Whitley anaerobic chamber (Microbiology International, Frederick, MD).

MBC. The MBC was determined with C. difficile ATCC 43255 at a concentration of 5×10⁵ cfu/mL. Oxadiazoles and the positive control antibiotics were serially diluted in supplemented brucella broth, and incubated anaerobically at 37° C. for 48 h. The cultures were plated on pre-reduced BHIS agar plates and bacterial colonies were counted. The MBC is the lowest concentration of the compound that gives ≥3-log₁₀ bacterial count reduction relative to the starting inoculum.

Time-kill assay. The compounds were added to pre-reduced supplemented Brucella broth at a concentration of 1×, 2×, 4×, and 8× MIC. An overnight culture of C. difficile ATCC43255 was added to the broth to a final concentration of 5×10⁶ CFU/mL and incubated at 37° C. anaerobically. Samples were collected for plate count at 0, 1, 3, 6, 9, 12, 24, and 48 h post incubation.

Post-antibiotic effect. The PAE for oxadiazole 57, VAN, and MTZ were determined using C. difficile ATCC43255 The bacteria were grown to ˜5×10⁶ CFU/mL in supplemented Brucella broth and the compounds were added at 1×, 2×, 4×, or 8× MIC and incubated at 37° C.′ for 1 h anaerobically. The cultures were diluted 1000-fold with fresh pre-warmed pre-reduced Brucella broth. Samples were taken at 0 h, 1 h pre-, 1 h post-dilution, and at every 2 h thereafter for viability counts. A control with no antibiotic was processed similarly. PAE was calculated as PAE=T−C, where T is the time the bacterial titer increases 1 log₁₀ relative to the post-dilution count and C is the time the titer increased 1 log₁₀ relative to the post-dilution titer in the control without antibiotic. The experiments were done in triplicate.

Emergence of resistance. This study was performed using C. difficile ATCC43255 in supplemented Brucella broth. Oxadiazole 57, MTZ, and VAN were diluted serially from 256× to 0.25× of their respective MIC values. A final concentration of 5×10⁶ CFU/mL of the bacteria was added before incubation at 37° C. anaerobically. Bacteria grown on the highest concentration of each compound from the previous day were used for the next inoculation. The experiment was conducted over 30 days, with serial daily passages. The bacteria from the highest concentration were tested for MIC after three passages in drug-free media.

Lactate dehydrogenase cytotoxicity assay. LDH cytotoxicity assay was evaluated using the Pierce LDH cytotoxicity assay kit (ThermoFisher Scientific, Waltham, MA) with THP-1 cells (ATCC TIB-202). For the LDH release assay, THP-1 cells were conditioned to grow in 5% fetal bovine serum. Briefly, 2×10⁴ cells/100 μL of medium were plated in triplicate in a 96-well tissue culture plate (Corning Incorporated, Corning, NY). Test compounds were twofold serially diluted with concentrations ranging from 256-0.25 μg/mL and the plates were incubated at 37° C. with 5% CO₂ for 24 h. Spontaneous LDH activity controls (water) and maximum LDH activity controls (10× lysis buffer) were also included. Following the 24-h incubation, 50 μL of each sample was transferred to another 96-well flat-bottom plate to perform the LDH assay. The absorbance at 490 nm and 680 nm were measured to determine the LDH activity. The remaining samples were used to perform a cell-viability assay using trypan-blue staining (Gibco, Life Technologies, Gaithersburg, MD).

XTT cytotoxicity assay. This assay was performed using XTT cell proliferation assay (Canvax, Spain) and HepG2 cells (ATCC HB-8065, ATCC, Manassas, VA) in triplicate, following the procedures previously reported (Eur J Med Chem. 2023, 253, 115329). The IC₅₀ values were calculated and analyzed by GraphPad Prism 5 (San Diego, CA).

### Example 2. Experiment Procedures of Synthesized Compounds

General procedure for syntheses of 4-cyanophenyl ethers and N′-hydroxybenzimidamides. We followed general literature methods for the preparation of 4-cyanophenyl ethers and N′-hydroxybenzimidamides reported in our previous work (ACS Med. Chem. Lett. 2020, 11, 322). The resulting amidoximes were used in the next step without further purification.

Oxadiazoles reported as anti-staphylococcal agents. The synthetic procedure and spectra of compounds that have been reported previously: compounds 9 and 71 by O'Daniel et al. (J. Am. Chem. Soc. 2014, 136, 3664); compounds 1, 10, 11, 12, 13, 14, 15, 16, 17, 18, 19, 20, 21, 22, 24, 27, 29, 31, 32, 34, 35, 36, 40, 41, 42, 43, 45, 46, 47 and 116 by Spink et al. (J. Med. Chem. 2015, 58, 1380); compounds 16, 25, 26, 28, 30, 37, 38, 39, 44 and 117 by Leemans et al. (Bioorg. Med. Chem. Lett. 2016, 26, 1011); compound 2 by Janardhanan et al. (Proc. Natl. Acad. Sci. USA. 2023, 120, e2304110120); compounds 72, 73, 74, 75, 76, 77, 83, 84, 85, 91, 94, 95, 96, 97, 98, 102, 103, 104 and 124 by Boudreau et al. (ACS Med. Chem. Lett. 2020, 11, 322); compounds 80, 81, 82, 100, 101, 105, 110, 111, 112, 113, 114, 122, 123, 127 and 128 by Ding et al. (Bioorg. Med. Chem. Lett. 2015, 25, 4854).

General procedure for the synthesis of 1,3,4-oxadiazoles (Method A). To an oven-dried flask were added carboxylic acid (1 equiv.) and carbodiimidazole (CDI, 1.1 equiv.) dissolved in anhydrous DMF (10 mL/mmol) under N₂. The mixture was stirred for 30 mins at room temperature. Then, amidoxime (1.0 equiv.) was added and the resulting mixture was stirred for an additional 3 h before heated to 140° C. for 8 h. The reaction was quenched with water (30 mL/mmol of reaction) and then washed with EtOAc (10 mL/mmol of reaction, 2×). The organic layers were combined and washed with water (3×), brine (1×), and dried over anhydrous Na₂SO₄. The solution was concentrated to dryness in vacuo, and purified by silica-gel chromatography (EtOAc:hexanes, 1:5-1:3) to give the desired product.

General procedure for the synthesis of 1,3,4-oxadiazoles (Method B). To an oven-dried flask were added carboxylic acid (1 equiv.), N,N′-dicyclohexylcarbodiimide (DCC, 1.1 equiv.) and amidoxime (1.0 equiv.) dissolved in anhydrous 1,4-dioxane (10 mL/mmol of reaction) under N₂. The mixture was heated at 100° C. for 12 h. The reaction was quenched with water (10 mL/mmol of reaction) and then washed with EtOAc (10 mL/mmol of reaction, 2×). The organic layers were combined and washed with water (2×), brine (1×), and the solution was dried over anhydrous Na₂SO₄, concentrated to dryness in vacuo, and purified by silica-gel chromatography (EtOAc:hexanes, 1:5-1:3) to give desired product.

General procedure for the synthesis of 1,3,4-oxadiazoles (Method C). To an oven-dried flask was added carboxylic acid (1 equiv.) dissolved in SOCl₂ (10 equiv.) under N₂. The mixture was heated to reflux for 1 h before drying in vacuo. The resulting residue (acyl chloride) was used directly in the following step.

The resulting acyl chloride (1 equiv) was dissolved in pyridine (10 mL/mmol of reaction), followed by the addition of amidoxime (1.1 mmol). The resulting mixture was stirred under N₂ and was heated to reflux for 12 h. The solvent was removed in vacuo and the crude product was purified by silica-gel chromatography (EtOAc:hexanes, 1:10-1:5) to afford the key intermediate. After removal of the solvent, the intermediate was dissolved in anhydrous DCM (10 mL/mmol of reaction) and cooled down to −78° C. in an acetone-dry ice bath. Two equivalents of BBr₃ (1 M in DCM) was added dropwise. The reaction was aged for 1 h before quenching with water (20 mL/mmol of reaction). The mixture was then allowed to warm up to room temperature and washed with DCM (20 mL/mmol of reaction, 2×). The organic layers were combined, dried over anhydrous Na₂SO₄ and filtered. The filtrate was concentrated in vacuo to dryness to give a residue, which was purified by silica-gel chromatography (EtOAc:hexanes, 1:5-1:3) to give desired product.

Deprotection of Boc group. Compounds 16, 53, 56 and 67 were synthesized from their Boc-protected precursors by a general deprotection method.

### Synthesis and Characterization of Synthesized Compounds.

(4-(3-(4-(4-(Trifluoromethyl)phenoxy)phenyl)-1,2,4-oxadiazol-5-yl)phenyl) methanamine hydrochloride salt (17). Method A followed by de-protection of Boc group; white solid; yield 85% (0.53 g, 1.03 mmol). ¹H NMR (400 MHz, MeOD) δ 8.31 (d, J=7.8 Hz, 2H), 8.21 (d, J=7.9 Hz, 2H), 7.84-7.59 (m, 4H), 7.37-7.10 (m, 4H), 4.26 (s, 2H). ¹³C NMR (126 MHz, MeOD) δ 175.42, 168.48, 159.84, 159.04, 138.37, 129.82, 129.42, 128.61, 127.35 (q, J_{CF}=3.8 Hz), 125.67 (q, J_{CF}=32.5 Hz), 124.76, 122.73, 124.45 (q, J_{CF}=270.4 Hz), 119.56, 118.98, 42.69. HRMS-ESI (m/z): [M+H]⁺ calcd for C₂₂H₁₇F₃N₃O₂, 412.1267; found, 412.1253.

5-(Pyridin-4-yl)-3-(4-(4-(trifluoromethyl)phenoxy)phenyl)-1,2,4-oxadiazole (23). Method A; white solid; yield 73% (0.40 g, 1.04 mmol). ¹H NMR (500 MHz, CDCl₃) δ 8.91-8.86 (m, 2H), 8.22-8.15 (m, 2H), 8.07-8.02 (m, 2H), 7.66-7.61 (m, 2H), 7.20-7.11 (m, 4H). ¹³C NMR (126 MHz, DMSO) δ 174.54, 168.63, 159.74, 159.04, 151.83, 130.92, 130.15, 128.34 (q, J=3.6 Hz), 125.06 (q, J=32.1 Hz), 124.84 (q, J=271.6 Hz), 122.29, 121.94, 120.46, 119.93. ¹⁹F NMR (376 MHz, DMSO) δ −60.35. HRMS-ESI (m/z): [M+H]⁺ calcd for C₂₀H₁₃F₃N₃O₂, 384.0954; found, 384.0958.

5-(1H-Pyrazol-4-yl)-3-(4-(4-(trifluoromethyl)phenoxy)phenyl)-1,2,4-oxadiazole (33). Method C; yellow solid; yield 32% (0.28 g, 0.75 mmol). ¹H NMR (400 MHz, CDCl₃) δ 8.21 (d, J=8.7 Hz, 2H), 8.04 (d, J=2.4 Hz, 1H), 7.65 (d, J=8.7 Hz, 2H), 7.23-7.12 (m, 5H). ¹³C NMR (101 MHz, CDCl₃) δ 171.03, 168.50, 159.50 (d, J_{CF}=1.1 Hz), 159.04, 137.67, 131.81, 129.82, 127.62 (q, J_{CF}=3.7 Hz), 126.17 (q, J_{CF}=32.9 Hz), 124.29 (q, J_{CF}=271.6 Hz), 122.67, 119.73, 119.19, 107.42. HRMS-ESI (m/z): [M+H]⁺ calcd for C₁₈H₁₂F₃N₄O₂, 373.0907; found, 373.0915.

5-(1H-Indol-2-yl)-3-(4-(4-(trifluoromethyl)phenoxy)phenyl)-1,2,4-oxadiazole (48). Method A; solid; yield 13% (46 mg, 0.11 mmol). ¹H NMR (500 MHz, CDCl₃) δ 9.13 (s, 1H), 8.29-8.07 (m, 2H), 7.75 (d, J=8.0 Hz, 1H), 7.64 (d, J=8.6 Hz, 2H), 7.53-7.43 (m, 2H), 7.41-7.33 (m, 1H), 7.25-7.19 (m, 1H), 7.19-7.08 (m, 4H). ¹³C NMR (126 MHz, CDCl₃) δ 170.21, 168.18, 159.58, 158.95, 137.70, 129.78, 128.10, 127.58 (q, J_{CF}=3.8 Hz), 126.11 (q, J_{CF}=32.9 Hz), 126.08, 124.29 (q, J=271.6 Hz), 122.75, 122.67, 121.70, 121.61, 119.74, 119.11, 112.03, 108.62. HRMS-ESI (m/z): [M+H]⁺ calcd for C₂₃H₁₅F₃N₃O₂, 422.1111; found, 422.1127.

4-(3-(4-(Cyclopentyloxy)phenyl)-1,2,4-oxadiazol-5-yl)benzoic acid (49). Method A followed with ester hydrolysis by LiOH; white solid; yield 44% (0.29 g, 0.83 mmol). ¹H NMR (400 MHz, DMSO) δ 13.42 (s, 1H), 8.27 (d, J=8.5 Hz, 2H), 8.16 (d, J=8.5 Hz, 2H), 8.00 (d, J=8.8 Hz, 2H), 7.08 (d, J=8.9 Hz, 2H), 4.97-4.86 (m, 1H), 2.03-1.86 (m, 2H), 1.80-1.65 (m, 4H), 1.65-1.52 (m, 2H). ¹³C NMR (101 MHz, DMSO) δ 174.82, 168.62, 166.88, 160.82, 135.08, 130.75, 129.30, 128.64, 127.37, 118.25, 116.44, 79.55, 32.74, 24.10. HRMS-ESI (m/z): [M+H]⁺ calcd for C₂₀H₁₉N₂O₄, 351.1339; found, 351.1346.

3-(4-(Cyclopentyloxy)phenyl)-5-(4-nitrophenyl)-1,2,4-oxadiazole (50). Method A; yellow solid; yield 75% (0.79 g, 2.24 mmol). ¹H NMR (400 MHz, CDCl₃) δ 8.40 (s, 4H), 8.07 (d, J=8.9 Hz, 2H), 6.99 (d, J=8.9 Hz, 2H), 4.92-4.77 (m, 1H), 2.00-1.77 (m, 6H), 1.71-1.61 (m, 2H). ¹³C NMR (126 MHz, CDCl₃) δ 173.5, 169.4, 161.1, 150.3, 129.9, 129.4, 129.3, 124.6, 118.3, 116.0, 79.8, 33.1, 24.3. HRMS-ESI (m/z): [M+H]⁺ calcd for C₁₉H₁₈N₃O₄, 352.1292; found, 352.1301.

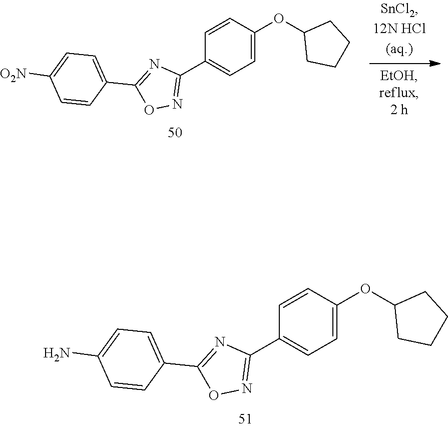 [SMILES: O=[N+]([O-])c1ccc(-c2nc(-c3ccc(OC4CCCC4)cc3)no2)cc1 ; Nc1ccc(-c2nc(-c3ccc(OC4CCCC4)cc3)no2)cc1]

4-(3-(4-(Cyclopentyloxy)phenyl)-1,2,4-oxadiazol-5-yl) aniline (51). To an oven-dried single-neck flask was added compound 50 (200 mg, 0.57 mmol) dissolved in 5 mL of ethanol. SnCl₂ (324 mg, 1.71 mmol) was added to the solution, followed by a few drops of 12N HCl (aq.). The mixture was brought to reflux for 2 h. The mixture was filtered and the solid was washed with ethanol. The filtrate was concentrated under reduced pressure and was purified by silica-gel column chromatography (EtOAc:hexanes=1:9 to 1:1) to give the title compound as an orange oil (162 mg, 89%, 0.51 mmol). ¹H NMR (400 MHz, CDCl₃) δ 8.08 (d, J=8.9 Hz, 2H), 8.01 (d, J=8.7 Hz, 2H), 6.98 (d, J=8.9 Hz, 2H), 6.76 (d, J=8.7 Hz, 2H), 4.91-4.75 (m, 1H), 4.18 (s, 2H), 2.04-1.75 (m, 6H), 1.74-1.55 (m, 2H). ¹³C NMR (126 MHz, CDCl₃) δ 175.9, 168.6, 160.6, 150.8, 130.2, 129.2, 119.4, 115.9, 114.7, 114.3, 79.7, 33.1, 24.3. HRMS-ESI (m/z): [M+H]⁺ calcd for C₁₉H₂₀N₃O₂, 322.1550; found, 322.1549.

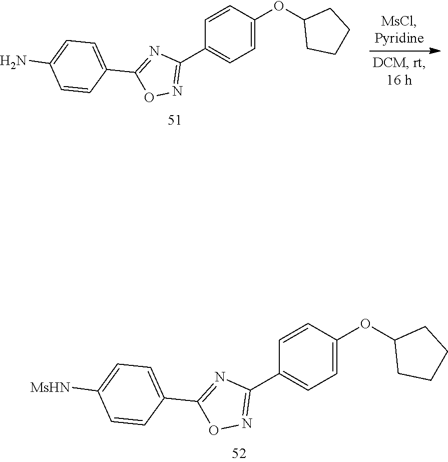

N-(4-(3-(4-(Cyclopentyloxy)phenyl)-1,2,4-oxadiazol-5-yl)phenyl) methanesulfonamide (52). To a solution of 51 (0.24 g, 0.76 mmol) and pyridine (26 μL, 1.36 mmol, 1.8 equiv.) in DCM (10 mL) was added MsCl (0.11 g, 0.98 mmol, 1.3 equiv) under an atmosphere of N₂. The mixture was stirred for 16 h before washing with 5% citric acid (aq.). The DCM fraction was dried over anhydrous Na₂SO₄ and filtered. The filtrate was concentrated in vacuo, and the residue was purified on a silica-gel column (EtOAc:hexanes=6:1-3:1) to give the desired product as a yellow solid (134 mg, 45%, 0.34 mmol). ¹H NMR (500 MHz, CDCl₃) δ 8.20 (d, J=8.8 Hz, 2H), 8.07 (d, J=8.9 Hz, 2H), 7.37 (d, J=8.8 Hz, 2H), 7.16 (s, 1H), 6.98 (d, J=8.9 Hz, 2H), 4.85 (tt, J=5.8, 2.7 Hz, 1H), 3.14 (s, 3H), 2.06-1.74 (m, 6H), 1.71-1.58 (m, 2H). ¹³C NMR (101 MHz, CDCl₃) δ 174.75, 169.03, 160.92, 141.03, 130.15, 129.29, 121.00, 119.26, 118.98, 115.99, 79.77, 40.36, 33.09, 24.28. HRMS-ESI (m/z): [M+H]⁺ calcd for C₂₀H₂₂N₃O₄S, 400.1326; found, 400.1333.

(4-(3-(4-(Cyclopentyloxy)phenyl)-1,2,4-oxadiazol-5-yl)phenyl) methanamine (53). Method A followed by de-protection of Boc group; white solid; yield 58% (0.38 g, 1.13 mmol). ¹H NMR (400 MHz, MeOD) δ 8.29 (d, J=8.3 Hz, 2H), 8.05 (d, J=8.9 Hz, 2H), 7.72 (d, J=8.2 Hz, 2H), 7.04 (d, J=8.9 Hz, 2H), 4.94-4.89 (m, 1H), 4.26 (s, 2H), 2.05-1.92 (m, 2H), 1.91-1.75 (m, 4H), 1.74-1.57 (m, 2H). ¹³C NMR (126 MHz, MeOD) δ 175.02, 168.88, 161.04, 138.21, 129.79, 128.84, 128.55, 124.89, 118.54, 115.71, 79.56, 42.70, 32.60, 23.81. HRMS-ESI (m/z): [M+H]⁺ calcd for C₂₀H₂₂N₃O₂, 336.1707; found, 336.1717.

3-(4-(Cyclopentyloxy)phenyl)-5-(indolin-5-yl)-1,2,4-oxadiazole (54). Method A followed by de-protection of Boc group; white solid; yield 55% (0.11 g, 0.29 mmol). ¹H NMR (500 MHz, DMSO) δ 7.98-7.90 (m, 2H), 7.81-7.75 (m, 2H), 7.08-7.02 (m, 2H), 6.69 (d, J=8.7 Hz, 1H), 4.93-4.86 (m, 1H), 3.59 (t, J=8.7 Hz, 2H), 3.06 (t, J=8.6 Hz, 2H), 1.99-1.90 (m, 2H), 1.77-1.63 (m, 4H), 1.63-1.53 (m, 2H). ¹³C NMR (126 MHz, DMSO) δ 176.24, 168.25, 160.72, 154.83, 131.68, 129.53, 129.33, 124.81, 119.15, 116.51, 113.55, 110.17, 79.69, 46.67, 32.94, 28.88, 24.30. HRMS-ESI (m/z): [M+H]⁺ calcd for C₂₁H₂₂N₃O₂, 348.1707; found, 348.1718.

5-(1H-Benzo[d]imidazol-6-yl)-3-(4-(cyclopentyloxy)phenyl)-1,2,4-oxadiazole (55). Method A; white solid; yield 64% (0.40 g, 1.16 mmol). ¹H NMR (500 MHz, DMSO) § 12.90 (s, 1H), 8.45 (s, 1H), 8.39 (brs, 1H), 8.06-7.92 (m, 3H), 7.81 (d, J=8.4 Hz, 1H), 7.05 (d, J=8.8 Hz, 2H), 4.95-4.78 (m, 1H), 2.01-1.86 (m, 2H), 1.77-1.63 (m, 4H), 1.63-1.47 (m, 2H). ¹³C NMR (101 MHz, DMSO) δ 176.41, 168.36, 160.63, 145.49, 129.21, 122.16, 118.66, 117.48, 116.32, 79.48, 32.72, 24.09. HRMS-ESI (m/z): [M+H]⁺ calcd for C₂₀H₁₉N₄O₂, 347.1503; found, 347.1510.

3-(4-(Cyclopentyloxy)phenyl)-5-(1H-indol-5-yl)-1,2,4-oxadiazole (56). Method A; solid; yield 63% (0.87 g, 2.51 mmol). ¹H NMR (400 MHz, DMSO) δ 11.63 (s, 1H), 8.46 (s, 1H), 8.01 (d, J=8.6 Hz, 2H), 7.91 (d, J=8.6 Hz, 1H), 7.63 (d, J=8.5 Hz, 1H), 7.55 (s, 1H), 7.09 (d, J=8.7 Hz, 2H), 6.68 (s, 1H), 5.01-4.79 (m, 1H), 2.04-1.86 (m, 2H), 1.81-1.66 (m, 4H), 1.66-1.54 (m, 2H). ¹³C NMR (126 MHz, DMSO) δ 177.3, 168.5, 160.8, 139.0, 129.4, 128.5, 128.4, 121.8, 121.2, 119.1, 116.6, 115.0, 113.1, 103.4, 79.7, 33.0, 24.3. HRMS-ESI (m/z): [M+H]⁺ calcd for C₂₁H₂₀N₃O₂, 346.1550; found, 346.1562.

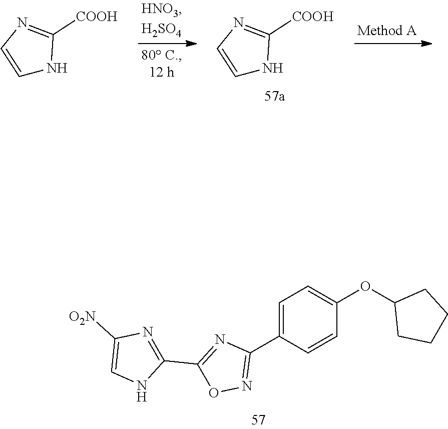 [SMILES: O=C(O)c1ncc[nH]1 ; O=C(O)c1ncc[nH]1 ; O=[N+]([O-])c1c[nH]c(-c2nc(-c3ccc(OC4CCCC4)cc3)no2)n1]

4-Nitro-1H-imidazole-2-carboxylic acid (57a). Sulfuric acid (27.5 mL, 517 mmol) was added to 1H-imidazole-2-carboxylic acid (5.00 g, 44.6 mmol) in an oven-dried single-neck round-bottom flask with stirring. Nitric acid (5 mL, 100 mmol) was then added dropwise. The mixture was stirred at 80° C. for 12 hr, followed by cooling to room temperature. The resulting mixture was added onto crushed ice dropwise. The white precipitate was then filtered and dried under vacuum overnight to give the title compound (3.5 g, 22 mmol, 50%) as a white solid. ¹H NMR (400 MHz, DMSO) δ 14.38 (s, 1H), 8.47 (s, 1H). ¹³C NMR (101 MHz, DMSO) δ 159.37, 147.85, 137.42, 122.17.

3-(4-(Cyclopentyloxy)phenyl)-5-(4-nitro-1H-imidazol-2-yl)-1,2,4-oxadiazole (57). Method A; white solid; yield 54% (0.70 g, 2.04 mmol). ¹H NMR (500 MHz, Acetone) δ 8.54 (s, 1H), 8.00 (AA′ BB′ d, J=8.9 Hz, 2H), 7.07 (AA′ BB′ d, J=8.9 Hz, 2H), 4.94 (tt, J=6.0, 2.5 Hz, 1H), 2.04-1.93 (m, 2H), 1.86-1.72 (m, 4H), 1.70-1.57 (m, 2H). ¹³C NMR (126 MHz, Acetone) δ 169.36, 167.20, 161.93, 149.92, 132.74, 129.79, 122.11, 118.66, 116.75, 80.27, 33.36, 24.62. HRMS-ESI (m/z): [M+H]⁺ calcd for C₁₆H₁₆N₅O₄, 342.1197; found, 342.1196.

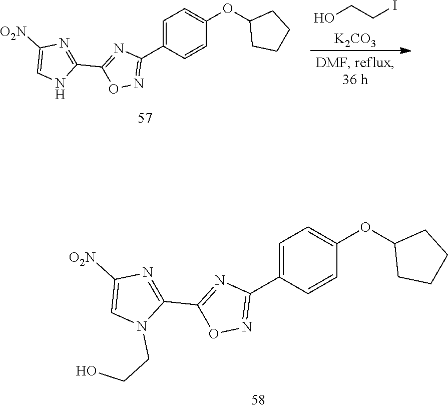

3-(4-(Cyclopentyloxy)phenyl)-5-(1-(2-hydroxyethyl)-4-nitro-1H-imidazol-2-yl)-1,2,4-oxadiazole (58). To a solution of compound 57 (320 mg, 0.94 mmol) and K₂CO₃ (389 mg, 2.81 mmol) in DMF (5 mL) was added 2-iodoethanol (88 μL, 1.13 mmol). The mixture was heated under reflux for 36 h. The mixture was diluted in water (15 mL) and washed with EtOAc (10 mL, 2×). The organic layers were combined, washed with brine, and dried over anhydrous Na₂SO₄. The suspension was filtered and the filtrate was evaporated to dryness. The residue was purified by silica-gel column chromatography (EtOAc:hexanes=1:1) to give the crude product as a yellow solid. The crude product was recrystallized in methanol to give the title compound (57 mg, 16%) as an off-white solid. ¹H NMR (500 MHz, DMSO) δ 8.79 (s, 1H), 8.01 (AA′BB′ d, J=8.9 Hz, 2H), 7.11 (AA′BB′ d, J=8.9 Hz, 2H), 5.06 (t, J=5.6 Hz, 1H), 4.96-4.90 (m, 1H), 4.73 (t, J=5.2 Hz, 2H), 3.86 (q, J=5.4 Hz, 2H), 2.02-1.92 (m, 2H), 1.77-1.66 (m, 4H), 1.66-1.55 (m, 2H). ¹³C NMR (126 MHz, DMSO) δ 168.43, 166.51, 161.26, 147.11, 131.95, 129.68, 127.34, 117.87, 116.74, 79.83, 60.17, 52.26, 32.94, 24.34. HRMS-ESI (m/z): [M+H]⁺ calcd for C₁₈H₂₀N₅O₅, 386.1459; found, 386.1444.

3-(4-(Cyclopentyloxy)phenyl)-5-(1-methyl-2-nitro-1H-imidazol-5-yl)-1,2,4-oxadiazole (59). Method A; white solid; yield 41% (0.23 g, 0.65 mmol). ¹H NMR (500 MHz, DMSO) δ 8.13 (s, 1H), 8.04-7.97 (m, 2H), 7.13-7.07 (m, 2H), 4.95-4.89 (m, 1H), 4.38 (s, 3H), 2.02-1.91 (m, 2H), 1.76-1.66 (m, 4H), 1.65-1.55 (m, 2H). ¹³C NMR (126 MHz, DMSO) δ 167.66, 166.49, 160.56, 148.21, 132.59, 128.97, 122.14, 117.20, 116.04, 79.15, 36.12, 32.27, 23.65. HRMS-ESI (m/z): [M+H]⁺ calcd for C₁₇H₁₈N₅O₄, 356.1353; found, 356.1364.

tert-Butyl (R)-(1-(3-(4-(cyclopentyloxy)phenyl)-1,2,4-oxadiazol-5-yl)ethyl) carbamate (60). Method B; white solid; yield 61% (0.31 g, 0.83 mmol). ¹H NMR (400 MHz, CDCl₃) δ 7.97 (d, J=8.7 Hz, 2H), 6.93 (d, J=8.7 Hz, 2H), 5.21 (brs, 1H), 5.18-5.06 (m, 1H), 4.86-4.77 (m, 1H), 2.01-1.72 (m, 6H), 1.72-1.62 (m, 2H), 1.61 (d, J=6.84 Hz, 3H), 1.46 (s, 9H). ¹³C NMR (101 MHz, DMSO-d₆) δ 20.19, 24.07, 28.31, 32.85, 44.25, 79.46, 80.46, 115.69, 118.42, 129.03, 154.79, 160.64, 168.06, 179.65. HRMS-ESI (m/z): [M+H]⁺ calcd for C₂₀H₂₈N₃O₄, 374.2074; found, 374.2082.

(R)-1-(3-(4-(Cyclopentyloxy)phenyl)-1,2,4-oxadiazol-5-yl) ethan-1-amine (61). Compound 60 (300 mg, 0.80 mol) and trifluoroacetic acid (0.92 mL, 12 mmol) were dissolved in anhydrous DCM (2 mL) in a 100-mL round-bottom flask. The resulting solution was stirred at 60° C. overnight. The solvent was removed in vacuo to give a light-yellow oil, which was taken up in ethyl acetate (20 mL). The solution was washed with saturated NaHCO₃, brine, and dried over anhydrous Na₂SO₄. After filtration, the filtrate was concentrated in vacuo to give a light-yellow oil, which was further purified by silica-gel column chromatography (ethyl acetate/hexane=1/1) to give the desired product (155 mg, 71% yield) as a colorless oil (overall yield 80%). ¹H NMR (400 MHz, CDCl₃) 7.95 (d, J=8.7 Hz, 1H), 6.90 (d, J=8.7 Hz, 1H), 4.80-4.73 (m, 1H), 4.30 (q, J=6.9 Hz, 1H), 1.99-1.68 (m, 8H, NH₂ peak overlapping, certified by the ¹H-NMR spectrum with aliquot D₂O), 1.65-1.57 (m, 2H), 1.55 (d, J=6.9 Hz, 3H). ¹H NMR (400 MHz, CDCl₃+aliquot D₂O) 7.94 (d, J=8.8 Hz, 2H), 6.90 (d, J=8.8 Hz, 2H), 4.81-4.70 (m, 1H), 4.28 (q, J=6.9 Hz, 1H), 1.95-1.68 (m, 6H), 1.66-1.56 (m, 2H), 1.54 (d, J=6.9 Hz, 3H). ¹³C NMR (101 MHz, CDCl₃) δ 21.6, 24.0, 32.8, 44.9, 79.4, 115.6, 118.6, 128.9, 160.5, 167.9, 182.7. HRMS-ESI (m/z): [M+H]⁺ calcd for C₁₅H₂₀N₃O₂, 274.1550; found, 274.1554.

5-((R)-1-((R)-2-(Boc-amino)propanamido)ethyl)-3-(4-(cyclopentyloxy)phenyl)-1,2,4-oxadiazole (62). To a 50-mL round-bottom flask were added compound 59 (150 mg, 0.55 mmol), Boc-D-alanine (104 mg, 0.55 mmol) and EDCI (158 mg, 0.82 mmol) in anhydrous DMF (5 mL). The resulting mixture was stirred at room temperature for 16 h. Water (20 mL) was added to quench the reaction. The aqueous layer was washed with EtOAc (10 mL, 2×). The organic layers were combined and washed with water, brine, and dried over anhydrous Na₂SO₄. The mixture was filtered and the filtrate was concentrated under reduced pressure to give a pale-yellow oil, which was further purified by silica-gel column chromatography (DCM:MeOH=98:2) to give the title compound (60 mg, 25%) as a colorless oil. ¹H NMR (400 MHz, CDCl₃) δ 7.96 (d, J=8.8 Hz, 2H), 6.99 (brs, 1H), 6.93 (d, J=8.8 Hz, 2H), 5.40 (quint, J=7.3 Hz, 1H), 5.03 (brs, 1H), 4.85-4.76 (m, 1H), 4.34-4.16 (m, 1H), 2.00-1.74 (m, 6H), 1.72-1.62 (m, 2H), 1.63 (d, J=7.1 Hz, 3H), 1.44 (s, 9H), 1.40 (d, J=7.1 Hz, 3H). ¹³C NMR (101 MHz, DMSO-d₆) δ 179.1, 172.4, 168.2, 160.8, 155.8, 129.2, 118.4, 115.8, 79.6, 50.1, 43.0, 42.9, 33.0, 28.4, 24.2, 19.9, 18.0. HRMS-ESI (m/z): [M+H]⁺ calcd for C₂₃H₃₂N₄O₅, 444.2367; found, 444.2386.

3-(4-(Cyclopentyloxy)phenyl)-5-(4-hydroxybenzyl)-1,2,4-oxadiazole (63). Method B; yellow solid; yield 36% (0.12 g, 0.36 mmol). ¹H NMR (400 MHz, CDCl₃) δ 7.98 (d, J=8.4 Hz, 2H), 7.17 (d, J=8.0 Hz, 2H), 7.01 (s, 1H), 6.95 (d, J=8.4 Hz, 2H), 6.76 (d, J=8.0 Hz, 2H), 4.94-4.69 (m, 1H), 4.20 (s, 2H), 2.02-1.73 (m, 6H), 1.73-1.54 (m, 2H). ¹³C NMR (126 MHz, CDCl₃) δ 178.8, 168.3, 160.9, 155.9, 130.4, 129.2, 125.1, 118.4, 116.2, 116.0, 79.8, 33.1, 32.4, 24.3. HRMS-ESI (m/z): [M+H]⁺ calcd for C₂₀H₂₁N₂O₃, 337.1547; found, 337.1557.

4-((3-(4-(Cyclopentyloxy)phenyl)-1,2,4-oxadiazol-5-yl)amino)phenol (64). To 5 mL of pyridine were added 5-chloro-3-(4-(cyclopentyloxy)phenyl)-1,2,4-oxadiazole (220 mg, 1 Eq, 831 μmol) and 4-aminophenol (99.8 mg, 1.1 Eq, 914 μmol). The mixture was stirred at room temperature for 48 h before removing the solvent in vacuo. The residue was taken up in ethyl acetate (10 mL) and washed with 10 mL 5% citric acid (aq.). The organic layer was dried over anhydrous Na₂SO₄ and filtered. The filtrate was concentrated in vacuo, and compound was purified by silica-gel column chromatography to give the title compound 64 (75 mg, 27%) as an off-white solid and the zincke reaction product 68 (142 mg, 70%) as a white solid. ¹H NMR (500 MHz, Acetone) δ 9.60 (s, 1H), 8.31 (s, 1H), 7.95 (AA′BB′ d, J=8.8 Hz, 2H), 7.57 (AA′BB′ d, J=8.9 Hz, 2H), 7.01 (AA′BB′ d, J=8.9 Hz, 2H), 6.89 (AA′BB′ d, J=8.9 Hz, 2H), 4.95-4.85 (m, 1H), 2.03-1.90 (m, 2H), 1.86-1.71 (m, 4H), 1.70-1.57 (m, 2H). ¹³C NMR (126 MHz, Acetone) δ 168.81, 167.94, 160.55, 153.85, 130.70, 128.74, 120.27, 119.99, 115.82, 115.71, 79.40, 32.72, 23.98. HRMS-ESI (m/z): [M+H]⁺ calcd for C₁₉H₂₀N₃O₃, 338.1499; found, 338.1485.

3-(4-(Cyclopentyloxy)phenyl)-1,2,4-oxadiazol-5-amine (68). ¹H NMR (500 MHz, Acetone) δ 7.85 (d, J=8.9 Hz, 2H), 7.11 (s, 2H), 6.97 (d, J=8.9 Hz, 2H), 4.95-4.82 (m, 1H), 2.02-1.89 (m, 2H), 1.86-1.70 (m, 4H), 1.69-1.55 (m, 2H). ¹³C NMR (126 MHz, Acetone) δ 172.30, 168.10, 160.35, 128.53, 120.33, 115.62, 79.35, 32.70, 23.95. HRMS-ESI (m/z): [M+H]⁺ calcd for C₁₃H₁₆N₃O₂, 246.1237; found, 246.1237.

3-(4-(Cyclopentyloxy)phenyl)-(E)-5-(3-hydroxystyryl)-1,2,4-oxadiazole (65). Method B; white solid; yield 19% (92 mg, 0.26 mmol). ¹H NMR (400 MHz, DMSO) δ 9.70 (s, 1H), 7.96 (d, J=8.9 Hz, 2H), 7.83 (d, J=16.4 Hz, 1H), 7.31 (d, J=16.4 Hz, 1H), 7.29-7.22 (m, 2H), 7.21-7.14 (m, 1H), 7.08 (d, J=8.9 Hz, 2H), 6.92-6.84 (m, 1H), 4.97-4.85 (m, 1H), 2.02-1.89 (m, 2H), 1.79-1.66 (m, 4H), 1.66-1.54 (m, 2H). ¹³C NMR (126 MHz, DMSO) δ 175.75, 168.41, 160.86, 158.46, 143.55, 136.22, 130.68, 129.38, 119.96, 118.82, 118.48, 116.59, 115.46, 110.79, 79.72, 32.95, 24.30. HRMS-ESI (m/z): [M+H]⁺ calcd for C₂₁H₂₁N₂O₃, 349.1547; found, 349.1547.

3-(4-(Cyclopentyloxy)phenyl)-(E)-5-(4-hydroxystyryl)-1,2,4-oxadiazole (66). Method B; yellow solid; yield 36% (0.17 g, 0.37 mmol). ¹H NMR (400 MHz, DMSO) δ 10.13 (s, 1H), 7.93 (d, J=7.5 Hz, 2H), 7.79 (d, J=16.3 Hz, 1H), 7.66 (d, J=7.3 Hz, 2H), 7.12 (d, J=16.3 Hz, 1H), 7.02 (d, J=7.5 Hz, 2H), 6.85 (d, J=7.3 Hz, 2H), 4.84 (s, 1H), 2.05-1.81 (m, 2H), 1.80-1.45 (m, 6H). ¹³C NMR (126 MHz, DMSO) δ 176.2, 168.3, 160.74, 160.72, 143.4, 131.0, 129.3, 126.1, 119.0, 116.6, 116.5, 107.1, 79.7, 32.9, 24.3. HRMS-ESI (m/z): [M+H]⁺ calcd for C₂₁H₂₁N₂O₃, 349.1547; found, 349.1551.

4-(3-(4-(Cyclopentyloxy)phenyl)-1,2,4-oxadiazol-5-yl)butan-1-amine hydrochloride salt (67) Method A followed with de-protection of Boc group by HCl(aq); white solid; yield 52% (0.41 g, 1.36 mmol). ¹H NMR (500 MHz, MeOD) δ 7.93 (d, J=8.9 Hz, 2H), 6.99 (d, J=8.9 Hz, 2H), 4.93-4.86 (m, 1H), 3.04 (t, J=7.3 Hz, 2H), 3.02-2.97 (m, 2H), 2.04-1.90 (m, 4H), 1.88-1.74 (m, 6H), 1.72-1.57 (m, 2H). ¹³C NMR (101 MHz, DMSO) δ 180.08, 167.69, 160.57, 129.09, 118.58, 116.35, 79.48, 38.63, 32.72, 26.73, 25.71, 24.08, 23.37. HRMS-ESI (m/z): [M+H]⁺ calcd for C₁₇H₂₄N₃O₂, 302.1863; found, 302.1865.

3-(4-(Cyclopentyloxy)phenyl)-1,2,4-oxadiazol-5 (4H)-one (69). To a solution of amidoxime (1.00 g, 4.54 mmol) in 1,4-dioxane (5.0 mL) were added CDI (N,N′-Carbonyldiimidazole) (5.45 mmol) and DBU (1,8-Diazabicyclo[5.4.0]undec-7-ene, 4.99 mmol). The reaction mixture was stirred at 100° C. for 3 h. The mixture was then diluted with water, and the pH was adjusted to 2.0 with 1 M HCl. The mixture was washed with EtOAc (2×). The combined organic layer was washed with water (50 mL, 3×), dried over anhydrous Na₂SO₄. The mixture was filtered and the filtrate was concentrated to dryness. The solid was collected and was triturated in cold EtOAc (3×3 mL) to give the title compound (0.82 g, 73%) as a white solid. ¹H NMR (500 MHz, DMSO) δ 7.70 (d, J=8.8 Hz, 2H), 7.08-7.02 (m, 2H), 4.90-4.86 (m, 1H), 1.98-1.89 (m, 2H), 1.71-1.66 (m, 4H), 1.60-1.55 (m, 2H). ¹³C NMR (126 MHz, DMSO) δ 161.28, 160.81, 157.81, 128.42, 116.61, 115.67, 79.85, 32.89, 24.30. HRMS-ESI (m/z): [M+H]⁺ calcd for C₁₃H₁₅N₂O₃, 247.1077; found, 247.1082.

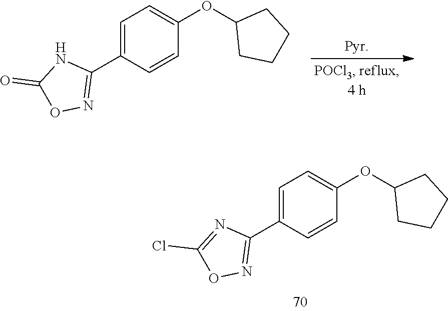 [SMILES: O=c1[nH]c(-c2ccc(OC3CCCC3)cc2)no1 ; Clc1nc(-c2ccc(OC3CCCC3)cc2)no1]

5-Chloro-3-(4-(cyclopentyloxy)phenyl)-1,2,4-oxadiazole (70). To an oven-dried single-neck flask was added 3-(4-(cyclopentyloxy)phenyl)-1,2,4-oxadiazol-5 (4H)-one (500 mg, 2.03 mmol) in POCl₃ (1.90 mL, 20.3 mmol), followed by the addition of pyridine (0.16 mL, 2.03 mmol). The mixture was refluxed for 4 h before, followed by cooling it to room temperature. POCl₃ was removed under reduced pressure to give a yellow residue, which was taken up in EtOAc:H₂O (1:1). The solution was washed with 5% citric acid, water (2×) and brine, dried over anhydrous Na₂SO₄. The mixture was filtered and the filtrate was concentrated under reduced pressure to give a pink residue, which was purified by silica-gel column chromatography (EtOAc:hexanes=1:10) to give the title compound (246 mg, 46%) as a colorless oil. ¹H NMR (400 MHz, CDCl₃) 7.92 (d, J=8.9 Hz, 2H), 6.93 (d, J=8.9 Hz, 2H), 4.84-4.76 (m, 1H), 1.99-1.73 (m, 6H) 1.69-1.55 (m, 2H). ¹³C NMR (101 MHz, CDCl₃) 170.0, 163.4, 161.1, 128.9, 117.4, 115.7, 79.5, 32.8, 24.1. HRMS-ESI (m/z): [M+H]⁺ calcd for C₁₃H₁₄ClN₂O₂, 265.0738; found, 265.0752.

4-(3-(4-((2-Bromocyclohex-2-en-1-yl)oxy)phenyl)-1,2,4-oxadiazol-5-yl)phenol (78). Method C; white solid; yield 46% (0.19 g, 0.48 mmol). ¹H NMR (400 MHz, DMSO) δ 8.41 (d, J=5.2 Hz, 1H), 8.09-8.02 (m, 2H), 7.83 (d, J=8.8 Hz, 2H), 7.80 (dt, J=5.2, 1.2 Hz, 1H), 7.67 (d, J=0.7 Hz, 1H), 7.44 (d, J=8.7 Hz, 2H), 7.02 (d, J=8.5 Hz, 2H). ¹³C NMR (101 MHz, DMSO) δ 176.33, 166.30, 163.08, 162.43, 156.75, 149.02, 137.93, 130.32, 127.17 (q, J=3.6 Hz), 125.29 (q, J=32.1 Hz), 124.20 (q, J=271.7 Hz), 121.96, 117.06, 116.44, 113.69, 109.45. HRMS-ESI (m/z): [M+H]⁺ calcd for C₂₀H₁₃F₃N₃O₃, 400.0904; found, 400.0917.

4-(3-(3-Bromo-4-phenoxyphenyl)-1,2,4-oxadiazol-5-yl)phenol (79). Method C; white solid; yield 88% (0.31 g, 0.76 mmol). ¹H NMR (500 MHz, DMSO) δ 10.60 (s, 1H), 8.31 (d, J=2.0 Hz, 1H), 8.05-7.99 (m, 3H), 7.49-7.42 (m, 2H), 7.24 (t, J=7.4 Hz, 1H), 7.12-7.07 (m, 3H), 6.99 (d, J=8.7 Hz, 2H). ¹³C NMR (126 MHz, DMSO) δ 176.37, 167.17, 162.85, 156.48, 156.08, 132.74, 131.06, 130.88, 128.89, 125.23, 123.77, 120.53, 119.52, 117.01, 114.62, 114.51. HRMS-ESI (m/z): [M+H]⁺ calcd for C₂₀H₁₄BrN₂O₃, 409.0182; found, 409.0195.

4-(3-(4-Ethoxyphenyl)-1,2,4-oxadiazol-5-yl)phenol (86). Method A; white solid; yield 87% (0.27 g, 0.96 mmol). ¹H NMR (400 MHz, DMSO) δ 10.52 (s, 1H), 8.00 (d, J=8.6 Hz, 2H), 7.97 (d, J=8.7 Hz, 2H), 7.09 (d, J=8.8 Hz, 2H), 6.97 (d, J=8.6 Hz, 2H), 4.10 (q, J=6.9 Hz, 2H), 1.34 (t, J=6.9 Hz, 3H). ¹³C NMR (126 MHz, DMSO) δ 175.86, 168.39, 162.63, 161.62, 130.71, 129.38, 119.21, 116.95, 115.62, 114.94, 64.03, 15.19. HRMS-ESI (m/z): [M+H]⁺ calcd for C₁₆H₁₅N₂O₃, 283.1077; found, 283.1076.

4-(3-(4-Propoxyphenyl)-1,2,4-oxadiazol-5-yl)phenol (87). Method A; white solid; yield 80% (0.35 g, 1.18 mmol). ¹H NMR (500 MHz, CDCl₃) δ 8.12 (d, J=8.8 Hz, 2H), 8.08 (d, J=8.9 Hz, 2H), 7.00 (d, J=8.9 Hz, 2H), 6.98 (d, J=8.8 Hz, 2H), 5.64 (s, 1H), 4.00 (t, J=6.6 Hz, 2H), 1.89-1.80 (m, 2H), 1.06 (t, J=7.4 Hz, 3H). ¹³C NMR (126 MHz, DMSO) δ 175.87, 168.39, 162.64, 161.79, 130.72, 129.39, 119.21, 116.95, 115.67, 114.94, 69.86, 22.63, 11.00. HRMS-ESI (m/z): [M+H]⁺ calcd for C₁₇H₁₇N₂O₃, 297.1234; found, 297.1229.

4-(3-(4-(Prop-2-yn-1-yloxy)phenyl)-1,2,4-oxadiazol-5-yl)phenol (88). Method A; pink solid; yield 49% (0.11 g, 0.37 mmol). ¹H NMR (400 MHz, DMSO) δ 10.57 (s, 1H), 8.07-7.97 (m, 4H), 7.23-7.13 (m, 2H), 7.04-6.96 (m, 2H), 4.91 (d, J=2.4 Hz, 2H), 3.65 (t, J=2.4 Hz, 1H). ¹³C NMR (101 MHz, DMSO) δ 175.77, 168.11, 162.47, 160.05, 130.57, 129.16, 119.85, 116.78, 115.97, 114.66, 79.30, 79.16, 56.10. HRMS-ESI (m/z): [M+H]⁺ calcd for C₁₇H₁₃N₂O₃, 293.0921; found, 293.0907.

4-(3-(4-(But-3-yn-1-yloxy)phenyl)-1,2,4-oxadiazol-5-yl)phenol (89). Method A; white solid; yield 36% (0.10 g, 0.33 mmol). ¹H NMR (500 MHz, Acetone) δ 8.25-7.90 (m, 4H), 7.22-6.98 (m, 4H), 4.22 (t, J=6.6 Hz, 2H), 2.72 (td, J=6.6, 2.6 Hz, 2H), 2.48 (t, J=2.5 Hz, 1H). ¹³C NMR (126 MHz, Acetone) δ 175.75, 168.39, 161.94, 161.27, 130.28, 129.06, 120.05, 116.34, 115.84, 115.13, 80.70, 70.65, 66.41, 19.20. HRMS-ESI (m/z): [M+H]⁺ calcd for C₁₈H₁₅N₂O₃, 307.1077; found, 307.1089.

4-(3-(4-Butoxyphenyl)-1,2,4-oxadiazol-5-yl)phenol (90). Method A; white solid; yield 77% (0.34 g, 1.10 mmol). ¹H NMR (500 MHz, CDCl₃) δ 8.11 (d, J=8.8 Hz, 2H), 8.08 (d, J=8.9 Hz, 2H), 7.03-6.96 (m, 4H), 5.79 (s, 1H), 4.03 (t, J=6.5 Hz, 2H), 1.87-1.74 (m, 2H), 1.57-1.45 (m, 2H), 0.99 (t, J=7.4 Hz, 3H). ¹³C NMR (126 MHz, DMSO) δ 175.88, 168.40, 162.65, 161.81, 130.74, 129.40, 119.19, 116.97, 115.70, 114.92, 68.11, 31.31, 19.38, 14.37. HRMS-ESI (m/z): [M+H]⁺ calcd for C₁₈H₁₉N₂O₃, 311.1390; found, 311.1381.

4-(3-(4-Cyclopropoxyphenyl)-1,2,4-oxadiazol-5-yl)phenol (92). Method A; white solid; yield 67% (0.21 g, 0.71 mmol). ¹H NMR (500 MHz, CDCl₃) δ 8.12 (d, J=8.9 Hz, 2H), 8.09 (d, J=9.0 Hz, 2H), 7.16 (d, J=8.9 Hz, 2H), 6.98 (d, J=8.9 Hz, 2H), 5.63 (s, 1H), 3.87-3.74 (m, 1H), 0.87-0.77 (m, 4H). ¹³C NMR (126 MHz, DMSO) δ 175.94, 168.40, 162.67, 161.80, 130.76, 129.36, 119.78, 116.99, 116.23, 114.89, 51.78, 6.65. HRMS-ESI (m/z): [M+H]⁺ calcd for C₁₇H₁₅N₂O₃, 295.1077; found, 295.1082.

4-(3-(4-Cyclobutoxyphenyl)-1,2,4-oxadiazol-5-yl)phenol (93). Method A; white solid; yield 55% (0.12 g, 0.39 mmol). ¹H NMR (500 MHz, DMSO) δ 10.53 (s, 1H), 8.20-7.74 (m, 4H), 7.09-6.77 (m, 4H), 4.87-4.61 (m, 1H), 2.48-2.40 (m, 2H), 2.16-1.94 (m, 2H), 1.85-1.74 (m, 1H), 1.71-1.57 (m, 1H). ¹³C NMR (126 MHz, DMSO) δ 175.89, 168.39, 162.65, 160.20, 130.74, 129.47, 119.31, 116.97, 116.07, 114.91, 71.67, 30.69, 13.46. HRMS-ESI (m/z): [M+H]⁺ calcd for C₁₈H₁₇N₂O₃, 309.1234; found, 309.1243.

4-(3-(4-((2-Bromocyclohex-2-en-1-yl)oxy)phenyl)-1,2,4-oxadiazol-5-yl)phenol (99). Method A; white solid; yield 83% (0.14 g, 0.34 mmol). ¹H NMR (500 MHz, DMSO) § 10.56 (s, 1H), 8.13-7.92 (m, 4H), 7.20 (d, J=8.9 Hz, 2H), 7.00 (d, J=8.7 Hz, 2H), 6.44 (dd, J=4.9, 3.2 Hz, 1H), 5.04 (s, 1H), 2.24-2.16 (m, 1H), 2.13-2.06 (m, 1H), 2.04-1.97 (m, 1H), 1.97-1.87 (m, 1H), 1.66-1.57 (m, 2H). ¹³C NMR (126 MHz, DMSO) δ 175.95, 168.32, 162.66, 160.70, 136.10, 130.76, 129.51, 120.82, 119.81, 116.97, 114.90, 75.87, 29.29, 27.94, 17.13. HRMS-ESI (m/z): [M+H]⁺ calcd for C₂₀H₁₈BrN₂O₃, 413.0495; found, 413.0512.

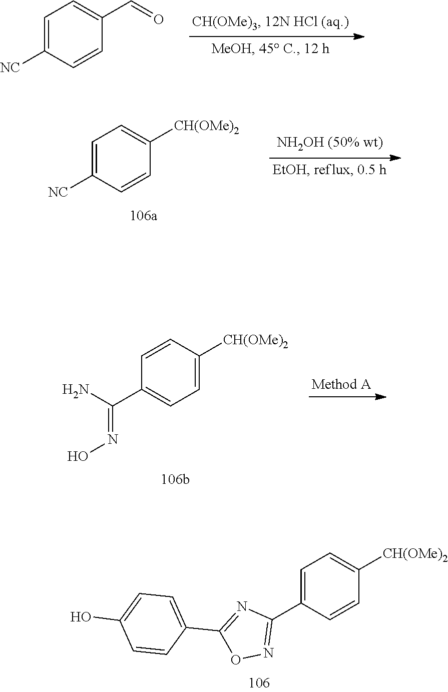

4-(Dimethoxymethyl)benzonitrile (106a). To a single-neck flask were added 4-formylbenzonitrile (2.9 g, 22.1 mmol) and trimethylorthoformate (14.1 g, 133.0 mmol) dissolved in MeOH (15 mL), followed by the addition of 12 N HCl (aq., 0.55 mL, 6.6 mmol). The mixture was heated for 16 h at 45° C. The mixture was then concentrated to dryness in vacuo. The residue was taken up in a mixture of MeOH and hexanes (20 mL, 1:1) and was washed with saturated Na₂CO₃ (aq.), and dried over anhydrous Na₂SO₄. The mixture was filtered and the filtrate was concentrated in vacuo to give the product (3.79 g) as a yellow powder. The product was used in the next step without further purification.

(Z)-4-(Dimethoxymethyl)-N′-hydroxybenzimidamide (106b). To a single-neck round-bottom flask charged with 20 mL of ethanol were added 4-(dimethoxymethyl)benzonitrile (2.78 g, 15.7 mmol) and hydroxylamine solution (4.85 mL, 78.4 mmol, aq., wt. 50%). The mixture was then heated to reflux for 20 mins. The resulting solution was concentrated to dryness in vacuo. The residue was purified by silica-gel column chromatography (100% EtOAc) to give the desired product as a yellow oil (3.29 g, 100%). ¹H NMR (400 MHz, CDCl₃) δ 9.30 (brs, 1H), 7.61 (d, J=8.3 Hz, 2H), 7.44 (d, J=8.2 Hz, 2H), 5.38 (s, 1H), 4.99 (s, 2H), 3.29 (s, 6H). ¹³C NMR (101 MHz, CDCl₃) δ 152.45, 139.77, 132.55, 127.03, 125.83, 102.57, 52.68.

4-(3-(4-(Dimethoxymethyl)phenyl)-1,2,4-oxadiazol-5-yl)phenol (106). Method A; white solid; yield 42% (0.27 g, 0.86 mmol). ¹H NMR (400 MHz, Acetone) δ 9.44 (brs, 1H), 8.15 (d, J=8.0 Hz, 2H), 8.10 (d, J=8.4 Hz, 2H), 7.62 (d, J=7.9 Hz, 2H), 7.09 (d, J=8.4 Hz, 2H), 5.48 (s, 1H), 3.32 (s, 6H). ¹³C NMR (126 MHz, Acetone) δ 176.74, 169.17, 162.67, 142.67, 131.01, 128.70, 128.25, 127.86, 117.03, 116.38, 103.24, 52.87. HRMS-ESI (m/z): [M+H]⁺ calcd for C₁₇H₁₇N₂O₄, 313.1183; found, 313.1178.

4-(5-(4-Hydroxyphenyl)-1,2,4-oxadiazol-3-yl)benzaldehyde (107). To a single-neck round-bottom flask was added 79 (500 mg, 1.6 mmol) dissolved in 5 mL of acetone, followed by a few drops of 12N HCl (aq.). The mixture was refluxed for 1 h. The mixture was concentrated to dryness and the residue was purified by silica-gel column chromatography (EtOAc:hexanes=1:1) to give the desired product (387 mg, 1.45 mmol, 91%) as a white solid. ¹H NMR (400 MHz, DMSO) δ 10.63 (s, 1H), 10.12 (s, 1H), 8.30 (d, J=8.1 Hz, 2H), 8.12 (d, J=8.1 Hz, 2H), 8.06 (d, J=8.6 Hz, 2H), 7.02 (d, J=8.6 Hz, 2H), 3.34 (s, 3H). ¹³C NMR (126 MHz, Acetone) δ 191.91, 168.05, 162.19, 138.71, 132.48, 130.44, 130.17, 128.04, 116.42, 115.46, 104.98. HRMS-ESI (m/z): [M+H]⁺ calcd for C₁₅H₁₁N₂O₃, 267.0764; found, 267.0764.

4-(3-(4-(2,2,2-Trifluoro-1-hydroxyethyl)phenyl)-1,2,4-oxadiazol-5-yl)phenol (108). To a single-neck round-bottom flask were added 80 (48 mg, 0.18 mmol) and trifluoromethyltrimethylsilane (31 mg, 0.22 mmol) dissolved in 2 mL of anhydrous THF at ice-water temperature. A catalytic amount of tetra-n-butylammonium fluoride (1.8 μmol) diluted with THF (2 mL) was then added dropwise to the reaction. The reaction was allowed to warm up to room temperature and was stirred for 1 h, followed by addition of more tetra-n-butylammonium fluoride (0.18 mmol). The resulting mixture was stirred for another 8 h at rt before being taken up in diethyl ether (10 mL) and washed with brine (10 mL, 3×). The organic layer was concentrated in vacuo to dryness. The residue was purified by silica-gel column chromatography (EtOAc:hexanes=1:10) to give the desired product (29 mg, 48%) as a white solid. ¹H NMR (500 MHz, Acetone) δ 9.44 (brs, 1H), 8.19 (AA′BB′ d, J=8.4 Hz, 2H), 8.11 (AA′BB′ d, J=8.8 Hz, 2H), 7.77 (AA′BB′ d, J=8.3 Hz, 2H), 7.09 (AA′BB′ d, J=8.8 Hz, 2H), 6.10 (s, 1H), 5.37 (q, J=7.1 Hz, 1H). ¹³C NMR (126 MHz, Acetone) δ 176.17, 168.38, 162.06, 139.07, 130.37, 128.51, 128.02, 127.31, 125.17 (q, J=281.9 Hz), 116.42, 115.70, 71.44 (q, J=30.8 Hz). ¹⁹F NMR (376 MHz, Acetone) δ −78.58. HRMS-ESI (m/z): [M+H]⁺ calcd for C₁₅H₁₁N₂O₃, 267.0764; found, 267.0764. HRMS-ESI (m/z): [M+H]⁺ calcd for C₁₆H₁₂F₃N₂O₃, 337.0795; found, 337.0807.

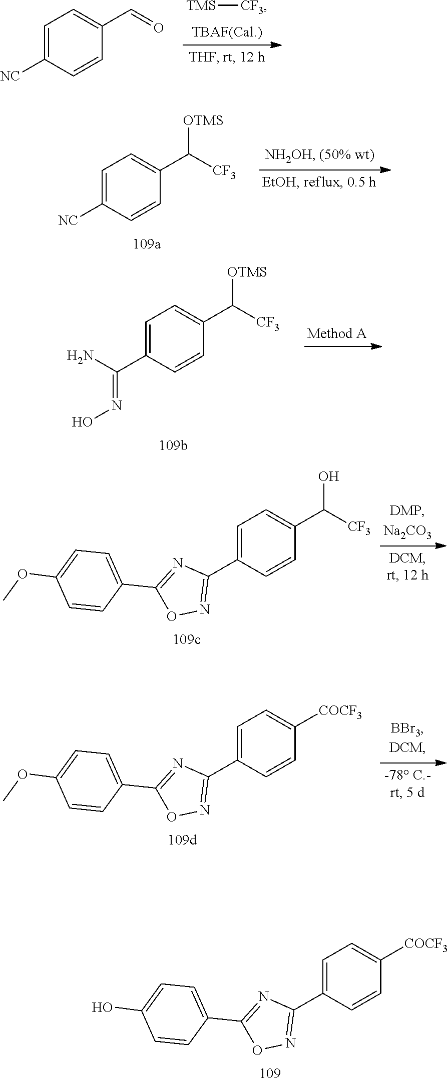

4-(2,2,2-Trifluoro-1-((trimethylsilyl)oxy)ethyl)benzonitrile (109a). To a single-neck round-bottom flask were added 4-formylbenzonitrile (1.20 g, 9.15 mmol) and trifluoromethyltrimethylsilane (1.56 g, 11.0 mmol) dissolved in 10 mL of anhydrous THF at ice-bath temperature. A catalytic amount of tetra-n-butylammonium fluoride (0.28 mmol) diluted with THF (10 mL) and was then added dropwise to the reaction mixture. The reaction was allowed to warm up to room temperature, at which point it was stirred for 12 h. The resulting mixture was taken up in diethyl ether (10 mL), washed with brine (10 mL, 3×), and dried over anhydrous Na₂SO₄. The mixture was filtered and the filtrate was concentrated to dryness in vacuo. The residue was purified by silica-gel column chromatography (100% hexanes) to give the desired product (2.50 g, >99%) as a yellowish oil. The crude product was used in further synthetic steps without further purification. ¹H NMR (400 MHz, CDCl₃) δ 7.71-7.66 (m, 2H), 7.58 (d, J=8.0 Hz, 2H), 4.96 (q, J=6.3 Hz, 1H), 0.14 (s, 9H).

(Z)-N′-Hydroxy-4-(2,2,2-trifluoro-1-((trimethylsilyl)oxy)ethyl)benzimidamide (109b). To a solution of 109a (500 mg, 1.83 mmol) in 20 mL of EtOH was added 1 mL of hydroxylamine solution (aq., 50% wt.). The mixture was refluxed for 1 h. The solution was concentrated to dryness in vacuo, and the solid was purified by silica-gel column chromatography (EtOAc:hexane=1:2) to give the crude product for the next synthetic step without further purification. ¹H NMR (400 MHz, CDCl₃) δ 9.12 (s, 1H), 7.57-7.48 (m, 2H), 7.37 (d, J=8.1 Hz, 2H), 4.96-4.70 (m, 3H), 0.00 (s, 9H).

5-(4-Methoxyphenyl)-3-(4-(2,2,2-trifluoro-1-hydroxyethyl)phenyl)-1,2,4-oxadiazole (109c). Method A, white solid (0.78 g, 61%). ¹H NMR (500 MHz, CDCl₃) δ 8.34-7.97 (m, 4H), 7.60 (d, J=7.3 Hz, 2H), 7.04 (d, J=7.0 Hz, 2H), 5.19-4.97 (m, 1H), 3.90 (s, 3H), 3.34 (brs, 1H). ¹³C NMR (126 MHz, CDCl₃) δ 176.08, 168.48, 163.54, 137.13, 132.52, 130.36, 128.18, 127.91, 124.33 (q, J=282.1 Hz), 116.78, 114.79, 72.66 (q, J=32.0 Hz), 55.76.

5-(4-Methoxyphenyl)-3-(4-(2,2,2-trifluoroacetyl)phenyl)-1,2,4-oxadiazole (109d). To a solution of 109c (1.00 g, 2.86 mmol) in 5 mL of DCM were added DMP (4.24 g, 9.99 mmol) and Na₂CO₃ (1.21 g, 11.42 mmol) at ice-water temperature. The mixture was brought to room temperature and was allowed to stir for 12 h. The resulting suspension was quenched by the addition of saturated Na₂S₂O₅ and washed with saturated Na₂CO; solution. The organic phase was collected, washed with brine (1×), and dried over anhydrous Na₂SO₄. After filtration, the filtrate was concentrated to dryness. The residue was purified by silica-gel column chromatography (EtOAc:hexanes=1:10) to give a solid, which was then triturated with cold MeOH to give the desired product (0.11 mg, 10%) as a white solid. ¹H NMR (500 MHz, CDCl₃) δ 8.36-8.30 (m, 2H), 8.19 (d, J=8.0 Hz, 2H), 8.16-8.11 (m, 2H), 7.07-7.01 (m, 2H), 3.90 (s, 3H). ¹³C NMR (126 MHz, CDCl₃) δ 180.22 (q, J_{CF}=35.5 Hz), 176.45, 167.77, 163.68, 133.82, 131.77, 130.73 (q, J_{CF}=1.9 Hz), 130.38, 128.23, 116.79 (d, J_{CF}=291.1 Hz), 116.57, 114.82, 55.76. HRMS-ESI (m/z): [M+H₃O]⁺ calcd for C₁₇H₁₄F₃N₂O₄, 367.0900; found, 367.0909.

5-(4-Hydroxyphenyl)-3-(4-(2,2,2-trifluoroacetyl)phenyl)-1,2,4-oxadiazole (109). To a solution of 109d (85 mg, 0.24 mmol) in 2 mL of DCM was added BBr₃ (1.0 M in heptane, 1.2 mL, 1.2 mmol) in a dry-ice-acetone bath. The mixture was allowed to warm up to room temperature and was allowed to stir for 19 days before quenching with saturated NH₄Cl (aq.). The resulting mixture was washed with brine, and dried over anhydrous Na₂SO₄. The mixture was filtered and the filtrate was concentrated to give the title compound (67 mg, 82%) as a white solid. ¹H NMR (500 MHz, Acetone) δ 8.40 (d, J=8.5 Hz, 2H), 8.29 (d, J=8.1 Hz, 2H), 8.11 (AA′BB′ d, J=8.7 Hz, 2H), 7.10 (AA′BB′ d, J=8.7 Hz, 2H). ¹³C NMR (126 MHz, Acetone) δ 176.72, 167.67, 162.29, 133.85, 131.90, 130.79 (q, J=1.9 Hz), 130.49, 128.19, 128.03 (q, J=284.9 Hz), 118.06, 116.45, 115.33. ¹⁹F NMR (376 MHz, Acetone) δ −72.38. HRMS-ESI (m/z): [M+H₃O]⁺ calcd for C₁₆H₁₂F₃N₂O₄, 353.0744; found, 353.0728.

5-(4-Nitro-1H-pyrazol-3-yl)-3-(4-(trifluoromethoxy)phenyl)-1,2,4-oxadiazole (115). Method C; white solid; yield 63% (0.9 g, 2.6 mmol). ¹H NMR (500 MHz, DMSO) δ 9.20 (s, 1H), 8.21 (d, J=8.2 Hz, 2H), 7.61 (d, J=8.0 Hz, 2H). ¹³C NMR (126 MHz, DMSO) δ 169.55, 167.99, 151.37, 136.07, 134.83, 133.12, 130.20, 125.46, 122.45, 120.65 (q, J=257.5 Hz). ¹⁹F NMR (376 MHz, DMSO) δ −56.66. HRMS-ESI (m/z): [M+H]⁺ calcd for C₁₂H₇F₃N₅O₄, 342.0445; found, 342.0433.

3-(Dibenzo[b,d]furan-2-yl)-5-(4-nitro-1H-pyrazol-3-yl)-1,2,4-oxadiazole (118). Method A; white solid; yield 84% (0.28 g, 0.81 mmol). ¹H NMR (400 MHz, DMSO) δ 9.24 (s, 1H), 8.93 (d, J=1.8 Hz, 1H), 8.38 (d, J=7.1 Hz, 1H), 8.26 (dd, J=8.6, 1.8 Hz, 1H), 7.95 (d, J=8.6 Hz, 1H), 7.79 (d, J=8.3 Hz, 1H), 7.65-7.56 (m, 1H), 7.48 (t, J=7.6 Hz, 1H). ¹³C NMR (101 MHz, DMSO) δ 168.67, 168.38, 157.28, 156.08, 134.16, 132.54, 131.11, 128.51, 126.75, 124.60, 123.69, 122.96, 121.95, 120.80, 120.67, 112.80, 111.90. HRMS-ESI (m/z): [M+H]⁺ calcd for C₁₇H₁₀N₅O₄, 348.0727; found, 348.0725.

3-(8-Methoxydibenzo[b,d]furan-2-yl)-5-(4-nitro-1H-pyrazol-3-yl)-1,2,4-oxadiazole (119). Method A; yellow solid; yield 48% (0.42 g, 1.11 mmol). ¹H NMR (400 MHz, DMSO) δ 9.23 (s, 1H), 8.91 (d, J=1.8 Hz, 1H), 8.22 (dd, J=8.6, 1.5 Hz, 1H), 7.97 (d, J=2.6 Hz, 1H), 7.87 (d, J=8.6 Hz, 1H), 7.66 (d, J=9.0 Hz, 1H), 7.15 (dd, J=9.0, 2.5 Hz, 1H), 3.88 (s, 3H). ¹³C NMR (126 MHz, DMSO) δ 169.29, 169.06, 158.60, 156.74, 151.29, 134.79, 133.13, 127.18, 125.62, 124.26, 121.46, 121.12, 117.21, 113.43, 113.08, 105.43, 56.49. HRMS-ESI (m/z): [M+H]⁺ calcd for C₁₈H₁₂N₅O₅, 378.0833; found, 378.0827.

3-(Benzofuran-5-yl)-5-(1H-indol-5-yl)-1,2,4-oxadiazole (120). Method A; white solid; yield 53% (0.23 g, 0.76 mmol). ¹H NMR (400 MHz, CDCl₃) δ 8.63 (s, 1H), 8.58 (d, J=1.6 Hz, 1H), 8.49 (d, J=1.7 Hz, 1H), 8.17 (dd, J=8.6, 1.7 Hz, 1H), 8.07 (dd, J=8.6, 1.7 Hz, 1H), 7.69 (d, J=2.2 Hz, 1H), 7.62 (d, J=8.6 Hz, 1H), 7.51 (d, J=8.5 Hz, 1H), 7.30 (dd, J=3.2, 2.4 Hz, 1H), 6.87 (dd, J=2.1, 0.7 Hz, 1H), 6.71 (ddd, J=3.1, 2.0, 0.9 Hz, 1H). ¹³C NMR (101 MHz, CDCl₃) δ 177.26, 169.31, 156.69, 146.24, 138.37, 128.24, 128.12, 126.17, 124.15, 122.54, 122.35, 122.21, 121.34, 116.49, 112.09, 111.92, 107.24, 104.30. HRMS-ESI (m/z): [M+H]⁺ calcd for C₁₈H₁₂N₃O₂, 302.0924; found, 302.0917.

3-(8-Fluorodibenzo[b,d]furan-2-yl)-5-(1H-indol-5-yl)-1,2,4-oxadiazole (121). Method A; yellow solid; yield 37% (0.11 g, 0.30 mmol). ¹H NMR (500 MHz, DMSO) δ 11.64 (s, 1H), 8.95 (s, 1H), 8.50 (s, 1H), 8.28 (dd, J=8.6, 1.7 Hz, 2H), 7.94 (d, J=8.5 Hz, 1H), 7.92 (d, J=8.6 Hz, 1H), 7.80 (dd, J=8.9, 4.0 Hz, 1H), 7.64 (d, J=8.6 Hz, 1H), 7.55 (d, J=2.3 Hz, 1H), 7.43 (td, J=9.1, 2.6 Hz, 1H), 6.68 (s, 1H). ¹³C NMR (101 MHz, DMSO) δ 177.72, 168.75, 159.43 (d, J=237.8 Hz), 158.75, 152.95, 139.12, 128.49 (d, J=5.5 Hz), 127.92, 125.01 (d, J=8.3 Hz), 124.94, 122.58, 121.98, 121.66, 121.27, 116.28 (d, J=26.1 Hz), 114.92, 113.75 (d, J=9.4 Hz), 113.43, 113.19, 108.79 (d, J=25.9 Hz), 103.41. HRMS-ESI (m/z): [M+H]⁺ calcd for C₂₂H₁₃FN₃O₂, 370.0986; found, 370.0987.

3-(2-Fluorophenyl)-5-(1H-indol-5-yl)-1,2,4-oxadiazole (125). Method A; solid; yield 69% (0.31 g, 1.11 mmol). ¹H NMR (500 MHz, CDCl₃) δ 8.59 (dt, J=1.5, 0.7 Hz, 1H), 8.46 (s, 1H), 8.21 (td, J=7.5, 1.8 Hz, 1H), 8.10-8.05 (m, 1H), 7.57-7.47 (m, 2H), 7.36-7.30 (m, 2H), 7.29-7.24 (m, 1H), 6.72 (ddd, J=3.1, 2.0, 0.9 Hz, 1H). ¹³C NMR (101 MHz, CDCl₃) δ 176.92, 165.90 (d, J=5.6 Hz), 161.04 (d, J=257.4 Hz), 138.42, 132.70 (d, J=8.3 Hz), 131.14 (d, J=2.3 Hz), 128.24, 126.15, 124.60 (d, J=3.7 Hz), 122.37 (d, J=19.0 Hz), 116.88 (d, J=21.3 Hz), 116.25, 115.96, 115.84, 111.92, 104.37. HRMS-ESI (m/z): [M+H]⁺ calcd for C₁₆H₁₁FN₃O, 280.0881; found 280.0890.

5-(1H-Indol-5-yl)-3-(4-nitrophenyl)-1,2,4-oxadiazole (126). Method A; solid; yield 81% (0.52 g, 1.70 mmol). ¹H NMR (500 MHz, DMSO) δ 11.64 (s, 1H), 8.48 (s, 1H), 8.45-8.39 (m, 2H), 8.37-8.31 (m, 2H), 7.91 (d, J=8.5 Hz, 1H), 7.62 (d, J=8.5 Hz, 1H), 7.54 (t, J=2.6 Hz, 1H), 6.67 (s, 1H). ¹³C NMR (101 MHz, DMSO) δ 178.30, 167.49, 149.81, 139.22, 133.08, 129.12, 128.57, 128.51, 125.13, 122.15, 121.29, 114.48, 113.25, 103.47. HRMS-ESI (m/z): [M+H]⁺ calcd for C₁₆H₁₁N₄O₃, 307.0826; found 307.0843.

5-(1H-Indol-5-yl)-3-(indolin-5-yl)-1,2,4-oxadiazole (129). Method A; white solid; yield 26% (83 mg, 0.27 mmol). ¹H NMR (500 MHz, CDCl₃) δ 8.55 (brs, 1H), 8.46 (s, 1H), 8.05 (dd, J=8.5, 1.6 Hz, 1H), 7.93 (brs, 1H), 7.89 (dd, J=8.1, 1.8 Hz, 1H), 7.51 (d, J=8.5 Hz, 1H), 7.32 (dd, J=3.5, 2.1 Hz, 1H), 6.74-6.66 (m, 2H), 4.07 (brs, 1H), 3.65 (d, J=8.4 Hz, 2H), 3.12 (t, J=8.5 Hz, 2H). ¹³C NMR (101 MHz, CDCl₃) δ 176.62, 169.35, 154.46, 138.22, 129.81, 128.17, 127.96, 126.02, 124.14, 122.24, 117.42, 116.78, 111.81, 108.80, 104.28, 47.60, 29.52. HRMS-ESI (m/z): [M+H]⁺ calcd for C₁₈H₁₅N₄O, 303.1240; found, 303.1235.

5-(4-Nitro-1H-imidazol-2-yl)-3-(4-(pyrrolidin-1-ylmethyl)phenyl)-1,2,4-oxadiazole Hydrochloride (130). Method A, followed by treatment of HCl (aq. 0.3 mL) in THF (2 mL) under reflux for 6 h to give the title compound as white solid; yield 51% (0.25 g, 0.65 mmol). Compound 130 crashed out in hot DMF, barely soluble in DMSO. Only ¹H NMR and HRMS of 130 were taken in this case. ¹H NMR (500 MHz, DMSO) δ 9.96 (brs, 1H), 8.15 (d, J=8.2 Hz, 2H), 7.92 (s, 1H), 7.71 (d, J=8.2 Hz, 2H), 4.41 (s, 2H), 3.24 (s, 4H), 1.94 (s, 4H). ¹³C NMR (126 MHz, DMSO) δ 181.40, 168.16, 150.27, 135.43, 132.00, 128.17, 127.97, 57.11, 53.80, 23.17. (The title compounds barely dissolves in DMSO at room temperature). HRMS-ESI (m/z): [M+H]⁺ calcd for C₁₆H₁₇N₆O₃, 341.1357; found, 341.1371.

3-(4-Cyclobutoxyphenyl)-5-(5-nitro-1H-imidazol-2-yl)-1,2,4-oxadiazole (131). Method A, followed by 12 N HCl (aq., 5 equiv.) treatment in methanol under reflux for 1 h to give the white suspension (removal of imidazole co-crystalized with the title compound). The suspension was filtered to give the title product as a white solid; yield 24% (100 mg, 0.31 mmol). ¹H NMR (500 MHz, DMSO) δ 8.73 (s, 1H), 8.00 (AA′BB′ d, J=8.8 Hz, 2H), 7.06 (AA′BB′ d, J=8.8 Hz, 2H), 4.79 (p, J=7.3 Hz, 1H), 2.49-2.43 (m, 2H), 2.08 (m, 2H), 1.86-1.75 (m, 1H), 1.73-1.60 (m, 1H). ¹³C NMR (126 MHz, DMSO) δ 168.64, 167.10, 160.59, 148.98, 132.35, 129.66, 123.62, 118.39, 116.27, 71.75, 30.68, 13.45. HRMS-ESI (m/z): [M+H]⁺ calcd for C₁₅H₁₄N₅O₄, 328.1040; found, 328.1027.

3-(4-((1-Methylpyrrolidin-3-yl)oxy)phenyl)-5-(5-nitro-1H-imidazol-2-yl)-1,2,4-oxadiazole hydrochloride (132). Method A, followed by treatment of HCl (aq. 0.3 mL) in THF (2 mL) under reflux for 6 h to give the title compound as white solid; yield 31% (0.16 g, 0.39 mmol). ¹H NMR (400 MHz, DMSO) δ 15.21 (s, 1H), 11.42 (s, 0.5H), 10.95 (s, 0.5H), 8.74 (s, 1H), 8.06 (d, J=8.8 Hz, 2H), 7.21 (d, J=8.6 Hz, 2H), 5.41-5.16 (m, 1H), 4.09-3.61 (m, 2H), 3.34-3.07 (m, 2H), 2.88 (s, 3H), 2.76-2.01 (m, 1H). ¹³C NMR (101 MHz, DMSO) δ 168.31, 166.98, 159.61, 148.76, 132.06, 129.54, 123.44, 119.03, 116.77, 75.99, 59.82, 53.83, 49.05, 41.59, 40.91, 30.78. HRMS-ESI (m/z): [M+H]⁺ calcd for C₁₆H₁₇N₆O₄, 357.1306; found, 357.1310.

3-(4-((1-(tert-Butoxycarbonyl) piperidin-4-yl)oxy)phenyl)-5-(5-nitro-1H-imidazol-2-yl)-1,2,4-oxadiazole (133). Method A; off-white solid; yield 69% (0.40 g, 0.88 mmol). ¹H NMR (400 MHz, DMSO) δ 8.90 (s, 0.3H), 8.27 (s, 1H), 7.99 (d, J=8.6 Hz, 2H), 7.61 (s, 0.7H), 7.17 (d, J=8.7 Hz, 2H), 4.69 (p, J=4.4 Hz, 1H), 3.77-3.58 (m, 2H), 3.28-3.09 (m, 2H), 2.03-1.88 (m, 2H), 1.65-1.48 (m, 2H), 1.41 (s, 9H). ¹³C NMR (101 MHz, DMSO) δ 169.90, 168.12, 160.03, 154.39, 149.65, 129.30, 128.59, 120.13, 118.99, 116.67, 79.24, 72.60, 40.59, 40.38, 40.17, 39.96, 39.75, 39.55, 39.34, 34.85, 30.83, 28.53. HRMS-ESI (m/z): [M+H]⁺ calcd for C₂₁H₂₅N₆O₆, 457.1830; found, 457.1815.

5-(4-Nitro-1H-imidazol-2-yl)-3-(4-(piperidin-4-yloxy)phenyl)-1,2,4-oxadiazole hydrochloride (134). To a solution of 133 (400 mg, 0.88 mmol) in THF (2 mL) was added 0.5 mL of 12 N HCl (aq.). The resulting solution was refluxed for 8 h. The white precipitate was filtered, rinsed with THF (1 mL, 2×), and dried under vacuum to give the title compound as a white solid (0.23 mg, 65% yield). ¹H NMR (500 MHz, DMSO) δ 9.17 (s, 2H), 8.70 (s, 1H), 8.00 (AA′BB′ d, J=8.9 Hz, 2H), 7.21 (AA′BB′ d, J=9.0 Hz, 2H), 4.79 (tt, J=7.3, 3.3 Hz, 1H), 3.26-3.17 (m, 2H), 3.15-3.00 (m, 2H), 2.26-2.06 (m, 2H), 1.96-1.79 (m, 2H). ¹³C NMR (126 MHz, DMSO) δ 168.55, 167.15, 160.15, 148.97, 132.34, 129.70, 123.68, 118.79, 117.00, 70.05, 41.13, 27.71. HRMS-ESI (m/z): [M+H]⁺ calcd for C₁₆H₁₇N₆O₄, 357.1306; found, 357.1303.

4-(3-(4-Phenoxyphenyl) isoxazol-5-yl)phenol (135). 4-Phenoxybenzaldehyde oxime (0.32 g, 1.50 mmol), 1-ethynyl-4-methoxybenzene (0.2 g, 1.5 mmol), 2,6-lutidine (0.16 g, 1.5 mmol) and NaI (0.23 g, 1.5 mmol) were added to 1,4-dioxane (10 mL) with stirring. Fresh 1-BuOCl (0.16 g, 1.5 mmol) was then added slowly. The resulting mixture was left to stir under an atmosphere of nitrogen for 24 h. The mixture was diluted with water (10 mL) and washed with DCM (10 mL, 3×). The organic layers were combined, dried over anhydrous Na₂SO₄. After filtration, the filtrate was concentrated to dryness to give the crude product for demethylation without further purification. The follow-up demethylation by BBr₃ (as described in Method C) gave the title compound as a white solid (0.21 g, 73% yield). ¹H NMR (400 MHz, CDCl₃) δ 7.82 (d, J=8.7 Hz, 2H), 7.74 (d, J=8.6 Hz, 2H), 7.38 (t, J=7.7 Hz, 2H), 7.24-7.12 (m, 1H), 7.12-7.04 (m, 4H), 6.96 (d, J=8.6 Hz, 2H), 6.67 (s, 1H). ¹³C NMR (101 MHz, CDCl₃) δ 169.28, 162.02, 157.13, 157.01, 158.54, 130.14, 128.61, 127.91, 124.16, 119.74, 118.87, 116.18, 109.12, 96.22, 89.59. HRMS-ESI (m/z): [M+H]⁺ calcd for C₂₁H₁₆NO₃, 330.1125; found, 330.1137.

4-(4-(4-Phenoxyphenyl)-1H-1,2,3-triazol-1-yl)phenol (136). 4-Azidophenol (210 mg, 1.55 mmol) and 1-ethynyl-4-phenoxybenzene (362 mg, 1.86 mmol) were added into [DBU]OAc (1 mL). Catalytic amount of CuBr (1 mol %) was added and the mixture was stirred at room temperature until it solidified. The resulting solid was purified by silica-gel column chromatography (EtOAc:hexanes=1:5) to afford the titled product (467 mg, 92%) as a white solid. ¹H NMR (500 MHz, DMSO) δ 9.99 (s, 1H), 9.08 (s, 1H), 7.96-7.90 (m, 2H), 7.75-7.68 (m, 2H), 7.42 (tt, J=7.5, 2.2 Hz, 2H), 7.20-7.15 (m, 1H), 7.14-7.10 (m, 2H), 7.09-7.05 (m, 2H), 7.00-6.93 (m, 2H). ¹³C NMR (126 MHz, DMSO) δ 158.46, 157.28, 157.09, 147.21, 130.82, 129.49, 127.73, 126.46, 124.38, 122.54, 119.83, 119.66, 119.52, 116.76. HRMS-ESI (m/z): [M+H]⁺ calcd for C₂₀H₁₆N₃O₂, 330.1237; found, 330.1228.

5-(4-(Cyclopentyloxy)phenyl)-3-(4-hydroxyphenyl)-1,2,4-oxadiazole (137). To an oven-dried 25-mL round-bottom flask were added 4-(cyclopentyloxy)benzoic acid (271 mg, 1.31 mmol), TBTU (422 mg, 1.31 mmol), HOBt (35.5 mg, 263 μmol) and DIPEA (510 mg, 687 μL, 3.94 mmol) dissolved in 2 mL of anhydrous DMF. The mixture was stirred at room temperature for 30 mins before (Z)-N′,4-dihydroxybenzimidamide (200 mg, 1.31 mmol) was added. The resulting mixture was stirred at room temperature for another 1 h. The mixture was then heated up to 120° C. for 24 h. After the mixture was allowed to cool down, the solution was diluted with 5% citric acid and washed with EtOAc (20 mL, 2×). The EtOAc layers were combined and washed with water (3×), 1 M NaOH (2×) and brine (2×). The organic phase was dried over anhydrous Na₂SO₄. The mixture was filtered and the filtrate was concentrated under reduced pressure to give a dark oil-like residue, which was purified by silica-gel column chromatography (EtOAc:hexanes=1:10) to give the title compound (162 mg, 503 μmol, 38%) as a yellow solid. ¹H NMR (400 MHz, CDCl₃) δ 8.03 (d, J=8.9 Hz, 2H), 7.94 (d, J=8.7 Hz, 2H), 6.90 (d, J=8.9 Hz, 2H), 6.86 (d, J=8.7 Hz, 2H), 4.81-4.69 (m, 1H), 1.95-1.64 (m, 6H), 1.63-1.47 (m, 2H). ¹³C NMR (126 MHz, CDCl₃) δ 175.80, 168.67, 162.17, 158.81, 130.26, 129.61, 119.44, 116.28, 116.12, 80.01, 33.07, 24.28. HRMS-ESI (m/z): [M+H]⁺ calcd for C₁₉H₁₉N₂O₃, 323.1390; found, 323.1381.

4-(Cyclopentyloxy)-N′-(4-hydroxybenzoyl)benzohydrazide (138). To a 25 mL flask were added 4-hydroxybenzohydrazide (200 mg, 1.31 mmol) and 4-(cyclopentyloxy)benzoic acid (271 mg, 1.31 mmol) and N-(3-(chlorodimethyl-15-azaneyl)propyl)-N-ethylmethanediimine (252 mg, 1.31 mmol) dissolved in anhydrous DMF (2 mL). The resulting mixture was stirred at 25° C. for 16 h. Water (10 mL) was added to the solution to give a white suspension, which was filtered and washed with water (3×) to give the title compound (420 mg, 1.23 mmol, 94%) as a white solid. ¹H NMR (500 MHz, DMSO) δ 7.85 (d, J=8.8 Hz, 2H), 7.75 (d, J=8.7 Hz, 2H), 6.97 (d, J=8.8 Hz, 2H), 6.78 (d, J=8.6 Hz, 2H), 4.94-4.85 (m, 1H), 1.98-1.86 (m, 2H), 1.75-1.63 (m, 4H), 1.62-1.50 (m, 2H). ¹³C NMR (126 MHz, DMSO) δ 166.23, 165.99, 161.02, 130.07, 129.94, 125.32, 115.85, 115.60, 79.64, 32.94, 24.32. HRMS-ESI (m/z): [M+H]⁺ calcd for C₁₉H₂₁N₂O₄, 341.1496; found, 341.1509.

4-(5-(4-(Cyclopentyloxy)phenyl)-1,3,4-oxadiazol-2-yl)phenol (139). To a 25 mL flask was added compound 138 (300 mg, 881 μmol) dissolved in a mixture of SOCl₂ (5 mL) and pyridine (1 mL). The resulting mixture was heated under reflux for 6 h. Residual SOCl₂ was distilled off to give a black oil, followed by the addition of a mixture of water (10 mL) and ethyl acetate (10 mL). The aqueous layer was washed with ethyl acetate (2×, 10 mL). The organic layers were combined and conc. in vacuo and further purified by a silica-gel column (ethyl acetate/hexane=1/10˜1/5) to give the titled compound (34 mg, 12%) as a white powder. ¹H NMR (400 MHz, DMSO-d₆) δ 10.33 (s, 1H), 8.00 (d, J=8.8 Hz, 2H), 7.94 (d, J=8.7 Hz, 2H), 7.11 (d, J=8.8 Hz, 2H), 6.97 (d, J=8.7 Hz, 2H), 4.94 (t, J=5.8 Hz, 1H), 2.05-1.89 (m, 2H), 1.83-1.66 (m, 4H), 1.66-1.54 (m, 2H). ¹³C NMR (101 MHz, DMSO-d₆) δ 24.10, 32.72, 79.64, 114.69, 115.92, 116.53, 116.62, 128.77, 129.00, 160.85, 161.17, 163.73, 164.15. HRMS-ESI (m/z): [M+H]⁺ calcd for C₁₉H₁₉N₂O₃, 323.1390; found, 323.1377.

### Example 3. Supplementary Minimal-Inhibitory Concentration Data

TABLE 3
Minimal-Inhibitory Concentrations (MICs) of oxadiazoles
against C. difficile and S. aureus cells.

| | | | S. aureus |
|---|---|---|---|
| | C. difficile ATCC 43255 | | ATCC 29213 |
| | MIC | MIC | MIC |
| Compound | (μg/mL) 24 h | (μg/mL) 48 h | (μg/mL) 24 h |
| 1 | 2 | 4 | 4 |
| 2 | 2 | 2 | 2 |
| 9 | 2 | 2 | 1 |
| 10 | 4 | 4 | 2 |
| 11 | 4 | 4 | 2 |
| 12 | >128 | >128 | 2 |
| 13 | 16 | 16 | >128 |
| 14 | >128 | >128 | >128 |
| 15 | >128 | >128 | >128 |
| 16 | 2 | 2 | 8 |
| 17 | 4 | 4 | 2-4 |
| 18 | >128 | >128 | >128 |
| 19 | >128 | >128 | ≥128 |
| 20 | >128 | >128 | 2 |
| 21 | >128 | >128 | >128 |
| 22 | >128 | >128 | >128 |
| 23 | >128 | >128 | |
| 24 | >128 | >128 | >128 |
| 25 | >128 | >128 | >128 |
| 26 | >128 | >128 | 1 |
| 27 | >128 | >128 | 1 |
| 28 | 128 | 128 | 2 |
| 29 | >128 | >128 | 4 |
| 30 | 4 | 4 | 2 |
| 31 | >128 | >128 | >128 |
| 32 | >128 | >128 | >128 |
| 33 | >128 | >128 | |
| 34 | 8 | 8 | 2 |
| 35 | >128 | >128 | 16 |
| 36 | >128 | >128 | >128 |
| 37 | >128 | >128 | 4 |
| 38 | >128 | >128 | >128 |
| 39 | >128 | >128 | >128 |
| 40 | >128 | >128 | >128 |
| 41 | >128 | >128 | >128 |
| 42 | >128 | >128 | >128 |
| 43 | >128 | >128 | >128 |
| 44 | >128 | >128 | >128 |
| 45 | >128 | >128 | >128 |
| 46 | >128 | >128 | >128 |
| 47 | 8 | 8 | 8 |
| 48 | >128 | >128 | |
| 49 | >128 | >128 | >128 |
| 50 | >128 | >128 | |
| 51* | 2 | >128 | 4 |
| 52 | >128 | >128 | >128 |
| 53 | 4 | 8 | 2 |
| 54* | 4 | 8 | |
| 55* | 128 | 128 | |
| 56 | >128 | >128 | |
| 57 | 0.125 | 0.25 | |
| 58 | >128 | >128 | |
| 59 | 32 | 32 | |
| 60 | >128 | >128 | |
| 61 | >128 | >128 | |
| 62 | >128 | >128 | |
| 63 | 8 | 16 | |
| 64 | 16 | 32 | |
| 65 | >128 | >128 | |
| 66 | 32 | 64 | |
| 67 | 128 | 128 | >128 |
| 68 | >128 | >128 | |
| 69 | 64 | 64 | |
| 70 | >128 | >128 | |
| 71 | 2 | 2 | 1 |
| 72 | 4 | 4 | 4 |
| 73 | 8 | 8 | 8 |
| 74 | 32 | 64 | 16 |
| 75 | >128 | >128 | >128 |
| 76 | 4 | 4 | 4 |
| 77* | 2 | 16 | 4 |
| 78 | >128 | >128 | |
| 79 | >128 | >128 | |
| 80* | 4 | >128 | |
| 81 | >128 | >128 | ≥128 |
| 82 | >128 | >128 | ≥128 |
| 83 | 64 | 128 | ≥128 |
| 84 | >128 | >128 | 32 |
| 85* | 4 | 64 | 1 |
| 86 | >128 | >128 | >128 |
| 87* | 4 | >128 | 128 |
| 88* | 16 | 128 | |
| 89 | >128 | >128 | |
| 90 | >128 | >128 | >128 |
| 91 | 64 | 64 | 16 |
| 92 | >128 | >128 | >128 |
| 93 | >128 | >128 | >128 |
| 94* | 8 | >128 | ≥128 |
| 95 | >128 | >128 | ≥128 |
| 96 | >128 | >128 | >128 |
| 97 | 64 | 64 | 16 |
| 98 | 64 | 64 | 16 |
| 99 | 2 | 2 | 4 |
| 100 | >128 | >128 | ≥128 |
| 101 | >128 | >128 | ≥128 |
| 102 | >128 | >128 | >128 |
| 103 | >128 | >128 | ≥128 |
| 104 | >128 | >128 | >128 |
| 105 | >128 | >128 | >128 |
| 106 | 64 | >128 | |
| 107 | 64 | >128 | |
| 108 | 32 | 32 | |
| 109 | 8 | 8 | |
| 110 | >128 | >128 | >128 |
| 111 | >128 | >128 | >128 |
| 112 | >128 | >128 | ≥128 |
| 113 | >128 | >128 | ≥128 |
| 114 | 64 | 64 | 8 |
| 115 | 2 | 4 | |
| 116 | >128 | >128 | 2 |
| 117 | 4 | 4 | 8 |
| 118 | 4 | 4 | 2 |
| 119* | 2 | 64 | 32 |
| 120 | >128 | >128 | 64 |
| 121 | >128 | >128 | |
| 122 | >128 | >128 | ≥128 |
| 123 | >128 | >128 | ≥128 |
| 124 | >128 | >128 | >128 |
| 125 | >128 | >128 | |
| 126 | >128 | >128 | |
| 127 | >128 | >128 | >128 |
| 128 | >128 | >128 | ≥128 |
| 129 | >128 | >128 | |
| 130 | >128 | >128 | |
| 131 | 0.5 | 2 | |
| 132 | >128 | >128 | |
| 133 | >128 | >128 | |
| 134 | >128 | >128 | |
| 135 | >128 | >128 | >128 |
| 136 | >128 | >128 | >128 |
| 137 | 4 | 4 | |
| 138 | >128 | >128 | |
| 139 | >128 | >128 | |

*C. difficile persisters observed

TABLE 4
MICs of oxadiazoles and reference antibiotics
against C. difficile strains.

| C. difficile strains | 1 | 2 | 54 | 57 | VAN | MTZ | FDX |
|---|---|---|---|---|---|---|---|
| BAA-1870 | 4 | 2 | 4 | 0.125 | 2 | 0.5 | 0.25 |
| BAA-1804 | 2 | 2 | 2 | 0.125 | 1 | 0.25 | 0.125 |
| BAA-1803 | 2 | 2 | 2 | 0.125 | 2 | 0.5 | 1 |
| BAA-1801 | 4 | 2 | 4 | 0.25 | 0.25 | 0.125 | 0.125 |
| BAA-1812 | 2 | 2 | 2 | 0.125 | 0.5 | 0.25 | 0.015 |
| BAA-1814 | 4 | 2 | 4 | 0.125 | 0.5 | 0.25 | 0.03 |
| ATCC43598 | 1 | 2 | 2 | 0.5 | 1 | 0.125 | 0.01 |
| NR49310 | 2 | 4 | 4 | 0.25 | 0.5 | 0.125 | ≤0.01 |
| NR49294 | 1 | 2 | 2 | 0.5 | 0.25 | 0.25 | 0.01 |
| NR49305 | 4 | 2 | 8 | 0.125 | 0.5 | 0.25 | 0.01 |
| ATCC43255 | 2 | 2 | 2 | 0.25 | 0.5 | 0.25 | 0.06 |
| NR49318 | 2 | 4 | 8 | 0.5 | 0.25 | 0.25 | 0.125 |
| NR49292 | 2 | 2 | 8 | 0.125 | 0.25 | 0.25 | 0.5 |
| NR49302 | 2 | 2 | 4 | 0.25 | 0.5 | 0.125 | 0.03 |
| RMA26675 | 4 | 4 | 8 | 2 | 1 | 4 | 4 |
| RMA27173 | 4 | 4 | 4 | 2 | 0.5 | 4 | 0.01 |
| RMA27081 | 2 | 4 | 8 | 1 | 1 | 0.5 | >8 |
| RMA23828 | 4 | 4 | 8 | 1 | 2 | 0.5 | >8 |
| RMA24923 | 4 | 4 | 2 | 0.125 | 8 | 0.25 | 0.06 |
| RMA24531 | 4 | 4 | 2 | 0.125 | 4 | 0.5 | 0.01 |
| RMA23691 | 4 | 4 | 2 | 1 | 4 | 0.5 | 4 |
| NR-13427 | 4 | 2 | 8 | 0.5 | 1 | 1 | 0.03 |
| NR-13428 | 8 | 4 | 8 | 0.5 | 2 | 1 | ≤0.03 |
| NR-13429 | 8 | 4 | 4 | 0.5 | 0.5 | 1 | 0.25 |
| NR-13430 | 8 | 2 | 8 | 0.5 | 0.5 | 0.5 | ≤0.03 |
| NR-13431 | 4 | 2 | 4 | 0.5 | 0.25 | 0.5 | 0.125 |
| NR-13433 | 4 | 4 | 4 | 1 | 1 | 0.5 | 0.125 |
| NR-13434 | 4 | 2 | 4 | 0.125 | 0.5 | 0.5 | 0.06 |
| NR-13435 | 4 | 4 | 4 | 0.5 | 0.5 | 0.5 | ≤0.03 |
| NR-13436 | 8 | 4 | 8 | 0.5 | 0.5 | 0.5 | 0.125 |
| NR-13437 | 8 | 4 | 8 | 0.5 | 0.5 | 0.5 | 0.25 |
| NR-13438 | 4 | 2 | 8 | 0.25 | 0.5 | 0.5 | 0.06 |
| NR-13553 | 4 | 2 | 4 | 0.125 | 0.5 | 0.5 | ≤0.03 |
| NR-32882 | 4 | 2 | 4 | 0.5 | 0.5 | 0.25 | 0.06 |
| NR-32883 | 4 | 2 | 4 | 0.5 | 0.5 | 0.5 | 0.125 |
| NR-32884 | 4 | 2 | 4 | 0.5 | 0.5 | 0.5 | 0.03 |
| NR-32885 | 8 | 4 | 8 | 0.5 | 1 | 0.5 | 0.25 |
| NR-32886 | 4 | 2 | 8 | 0.5 | 0.5 | 0.5 | 0.03 |
| NR-32887 | 8 | 4 | 4 | 1 | 0.5 | 2 | 0.125 |
| NR-32888 | 8 | 4 | 8 | 1 | 0.5 | 0.5 | 0.03 |
| NR-32889 | 4 | 4 | 4 | 1 | 2 | 2 | 0.25 |
| NR-32890 | 4 | 2 | 4 | 0.125 | 2 | 0.5 | 0.06 |
| NR-32891 | 8 | 4 | 4 | 0.5 | 1 | 0.25 | 0.03 |
| NR-32892 | 4 | 4 | 4 | 0.25 | 0.5 | 0.5 | 0.03 |
| NR-32895 | 2 | 2 | 8 | 0.25 | 2 | 1 | 0.03 |
| NR-32896 | 2 | 2 | 4 | 0.25 | 2 | 1 | 0.03 |
| NR-32897 | 8 | 2 | 4 | 0.25 | 0.25 | 0.5 | 0.03 |
| NR-32900 | 8 | 4 | 4 | 0.25 | 1 | 1 | 0.03 |
| NR-32903 | 8 | 2 | 2 | 0.25 | 0.5 | 0.5 | 0.03 |
| NR-32904 | 4 | 2 | 4 | 0.125 | 0.25 | 0.5 | 0.03 |
| NR-49277 | 4 | 2 | 4 | 0.5 | 0.5 | 0.5 | 0.06 |
| NR-49278 | 4 | 2 | 4 | 0.5 | 0.5 | 0.5 | 0.25 |
| NR-49279 | 4 | 2 | 4 | 0.5 | 0.5 | 1 | 0.25 |
| NR-49280 | 8 | 4 | 2 | 0.25 | 1 | 1 | 0.125 |
| NR-49281 | 4 | 4 | 4 | 0.25 | 0.5 | 1 | 0.25 |
| NR-49282 | 8 | 4 | 2 | 0.5 | 0.5 | 0.5 | 0.06 |
| NR-49283 | 8 | 4 | 2 | 0.5 | 0.5 | 0.5 | 0.03 |
| NR-49284 | 8 | 4 | 4 | 0.5 | 0.5 | 0.5 | 0.06 |
| NR-49285 | 4 | 2 | 4 | 0.5 | 0.25 | 0.5 | 0.06 |
| NR-49286 | 4 | 2 | 2 | 0.5 | 0.5 | 1 | 0.25 |
| NR-49287 | 4 | 2 | 4 | 0.25 | 0.5 | 0.5 | 0.25 |
| NR-49288 | 4 | 2 | 4 | 1 | 0.5 | 0.5 | 0.25 |
| NR-49289 | 8 | 2 | 4 | 1 | 0.5 | 0.5 | 0.03 |
| NR-49290 | 8 | 4 | 4 | 1 | 0.5 | 1 | 0.06 |
| NR-49291 | 8 | 2 | 4 | 1 | 1 | 1 | 0.25 |
| NR-49293 | 4 | 2 | 2 | 0.5 | 0.5 | 0.5 | ≤0.03 |
| NR-49295 | 4 | 4 | 4 | 1 | 0.5 | 0.5 | ≤0.03 |
| NR-49296 | 4 | 4 | 8 | 1 | 0.5 | 0.5 | ≤0.03 |
| NR-49297 | 4 | 2 | 4 | 1 | 0.5 | 0.5 | 0.0125 |
| NR-49298 | 4 | 4 | 8 | 1 | 1 | 1 | 0.125 |
| NR-49299 | 4 | 2 | 8 | 1 | 0.5 | 0.5 | 0.125 |
| NR-49300 | 4 | 2 | 8 | 1 | 0.5 | 0.5 | 0.03 |
| NR-49301 | 4 | 2 | 8 | 0.5 | 0.5 | 0.5 | 0.03 |
| NR-49303 | 4 | 2 | 4 | 1 | 0.5 | 0.5 | 0.03 |
| NR-49304 | 4 | 4 | 8 | 1 | 0.5 | 0.5 | 0.06 |
| NR-49306 | 4 | 4 | 4 | 0.5 | 1 | 0.5 | 0.03 |
| NR-49307 | 4 | 4 | 2 | 0.5 | 1 | 0.5 | 0.03 |
| NR-49308 | 8 | 2 | 2 | 0.5 | 0.5 | 0.5 | 0.125 |
| NR-49309 | 4 | 2 | 8 | 0.5 | 1 | 0.25 | 0.03 |
| NR-49311 | 4 | 2 | 4 | 1 | 0.5 | 0.5 | 0.03 |
| NR-49312 | 8 | 4 | 4 | 0.5 | 0.5 | 0.5 | 0.125 |
| NR-49313 | 4 | 2 | 4 | 0.5 | 0.5 | 0.5 | 0.03 |
| NR-49314 | 4 | 2 | 4 | 0.5 | 0.5 | 0.5 | 0.25 |
| NR-49315 | 4 | 4 | 2 | 0.5 | 0.5 | 0.5 | 0.06 |
| NR-49316 | 4 | 4 | 4 | 1 | 0.5 | 0.5 | 0.125 |
| NR-49317 | 8 | 4 | 2 | 1 | 0.5 | 0.5 | 0.06 |
| NR-49319 | 8 | 4 | 4 | 0.25 | 0.5 | 0.5 | 0.125 |
| NR-49320 | 4 | 2 | 4 | 0.5 | 1 | 0.5 | 0.125 |
| NR-49321 | 8 | 4 | 4 | 1 | 0.25 | 0.5 | 0.125 |
| NR-49323 | 8 | 4 | 4 | 0.5 | 0.5 | 0.25 | ≤0.03 |
| NR-49324 | 8 | 4 | 8 | 1 | 0.5 | 0.25 | 0.06 |
| NR-49325 | 8 | 4 | 4 | 0.25 | 0.5 | 0.5 | 0.03 |
| NR-49326 | 4 | 2 | 8 | 0.5 | 0.5 | 0.5 | 0.03 |
| NR-49327 | 4 | 2 | 8 | 0.25 | 0.5 | 0.5 | 0.03 |
| NR-49328 | 4 | 2 | 8 | 2 | 0.25 | 0.25 | 0.03 |
| NR-49329 | 4 | 2 | 8 | 0.5 | 0.25 | 0.25 | 0.03 |
| HM-88 | 8 | 4 | 4 | 1 | 0.5 | 0.25 | 0.03 |
| HM-89 | 8 | 4 | 8 | 0.5 | 0.25 | 0.25 | 0.03 |
| HM-745 | 8 | 4 | 2 | 0.5 | 0.5 | 0.5 | 0.125 |
| HM-746 | 4 | 4 | 2 | 0.5 | 0.5 | 0.25 | ≤0.03 |
| HM-747 | 8 | 4 | 1 | 0.25 | 0.5 | 0.5 | 0.06 |
| MIC₅₀ | 4 | 2 | 4 | 0.5 | 0.5 | 0.5 | 0.06 |
| MIC₉₀ | 8 | 4 | 8 | 1 | 2 | 1 | 0.25 |

### Example 4. Spore Germination Inhibition Assay and Data

Assays for determining spore germination inhibition were carried out as follows.

C. difficile strain ATCC43255 was used for this assay. Spores were prepared and purified as described previously (Sorg et al. (2010) J. Bacteriology 192 (19): 4983-4990) and spore stocks were stored at 4° C. until use. Spores (1 mL of OD₆₀₀=0.3-0.4, equivalent to ˜10⁷ CFU/mL) were incubated in 10% brain heart infusion supplemented (BHIS) medium inside a cuvette with or without germinant (6 mM taurocholate and 12 mM glycine). Compound was added to a final concentration of 45 μM. The OD₆₀₀ values were read every 10 min over a period of 60 min, when almost all spores had germinated. The initiation of spore germination, indicating a conversion from phase-bright to phase-dark spores was detected as a loss in OD₆₀₀. The assay was done in duplicate.

TABLE 5
C. difficile Inhibition Data for Select Compounds.

| | | C. difficile Inhibition | |
|---|---|---|---|
| | Compound | ATCC43255 MIC | % Spore Germination |
| Entry | Code | (mg · L⁻¹) | Inhibition at 45 μM |
| 1 | 57 | 0.25 | <10 |
| 2 | DR-04-089 | >128 | 20 ± 3 |
| 3 | DR-03-138 | >128 | 30 ± 14 |
| 4 | YQ-4-161 | >128 | 55 ± 1 |
| 5 | YQ-4-163 | 16 | 68 ± 5 |
| 6 | YQ-4-104 | 8 | 74 ± 5 |
| 7 | YQ-4-107 | | 24 ± 3 |
| 8 | YQ-4-164 | 64 | 24 ± 6 |
| 9 | YQ-4-165 | 8 | 50 ± 2 |
| 10 | YQ-4-81 | 4 | 41 ± 1 |
| 11 | YQ-4-99 | 8 | 44 ± 3 |
| 12 | YQ-4-167 | >128 | 80 ± 1 |
| 13 | YQ-4-169 | 8 | 63 ± 3 |
| 14 | YQ-4-170 | 8 | 48 ± 2 |
| 15 | MAB-01-140A | >128 | 57 ± 3 |
| 16 | MAB-01-250A | >128 | 62 ± 2 |
| 17 | MAB-02-27A | >128 | 17 ± 1 |
| 18 | MAB-02-29A | >128 | 26 ± 1 |
| 19 | MAB-02-59A | >128 | 30 ± 3 |
| 20 | DR-05-250 | >128 | 73 ± 5 |
| 21 | YQ-4-155 | 16 | 33 ± 1 |
| 22 | DR-06-025 | >128 | 44 ± 2 |

The percentage of spore germination inhibition (SGI) shown in Table 5 was calculated by the formula:

$$SGI = \frac{Ungerminated spores}{Total spores} = ( 1 - \frac{Δ {OD}_{600} ( Treated ) - Δ {OD}_{600} ( Background )}{Δ {OD}_{600} ( Untreated )} \times 100 \% .$$

### Example 5. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of formula I or II: 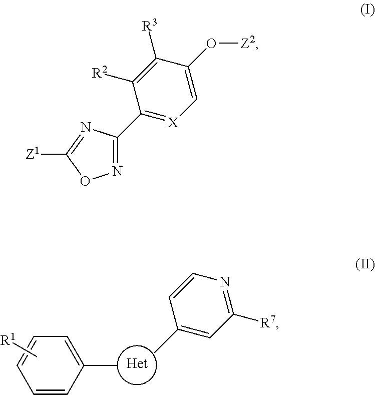
or a pharmaceutically acceptable salt thereof;
wherein,
Het is a 1,2,4-oxadiazole;
R¹ is aminoalkyl or OH;
R² is H or NH₂;
R³ is H, CF₃, or 3-(trifluoromethyl)-3H-diazirine-3-yl;
X is CH or N;
Z¹ is nitro-imidazole, nitro-pyrazole, pyrrolidinone, 4-(aminoalkyl)phenyl, 4-hydroxylphenyl, or aminoalkyl;
Z² is cyclopentyl or —(C₃-C₄ or C₆)cycloalkyl, branched or unbranched —(C₁-C₆)alkyl, or Ar wherein Ar is: 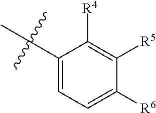
wherein,
R⁴ is H, CH₂(halo), or NO₂;
R⁵ is H or halo; and
R⁶ is H, halo, CF₃, —C(═O)CH₃, or —C≡CH; and
R⁷ is Ar or OAr; and
provided Z² is not cyclopentyl when Z¹ is 4-(aminomethyl)phenyl or 4-hydroxylphenyl.

2. The compound of claim 1 wherein Z¹ is 5-nitro-1H-imidazole-2-yl, —(CH₂)₄NH₂, 4-hydroxyphenyl, 4-(NH₂CH₂)phenyl, 4-(CH₃NHCH₂)phenyl, 4-nitro-1H-pyrazole-3-yl, or pyrrolidin-2-one-4-yl.

3. The compound of claim 1 wherein Z² is cyclopentyl, cyclopropyl, cyclobutyl, or cyclohexyl.

4. The compound of claim 1 wherein the compound is 57: 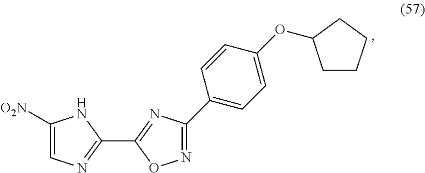 [SMILES: O=[N+]([O-])c1cnc(-c2nc(-c3ccc(OC4CCCC4)cc3)no2)[nH]1]
or a pharmaceutically acceptable salt thereof.

5. The compound of claim 1 wherein Z² is 4-(trifluoromethyl)phenyl, 4-fluorophenyl, 3,4-difluorophenyl, 4-iodophenyl, 3-iodophenyl, 2-nitrophenyl, 2-(bromomethyl)phenyl, phenyl, 4-acetophenyl, or 4-phenylethyne.

6. The compound of claim 1 wherein formula I is represented by formula III: 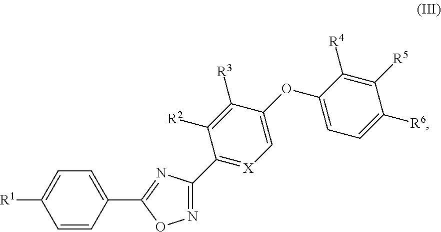
or a pharmaceutically acceptable salt thereof;
wherein,
R¹ is aminoalkyl or OH;
R² is H or NH₂;
R³ is H, CF₃, or 3-(trifluoromethyl)-3H-diazirine-3-yl;
R⁴ is H, —CH₂(halo), or NO₂;
R⁵ is H or halo;
R⁶ is H, halo, CF₃, —C(═O)CH₃, or —C≡CH; and
X is CH or N.

7. The compound of claim 6 wherein R¹ is —CH₂NH₂, —CH₂NHCH₃, or —CH₂CH₂NH₂.

8. The compound of claim 6 wherein R⁶ is CF₃.

9. The compound of claim 6 wherein R² and R³ are H.

10. The compound of claim 6 wherein R⁴ and R⁵ are H.

11. The compound of claim 1 wherein formula II is represented by formula IV or V: 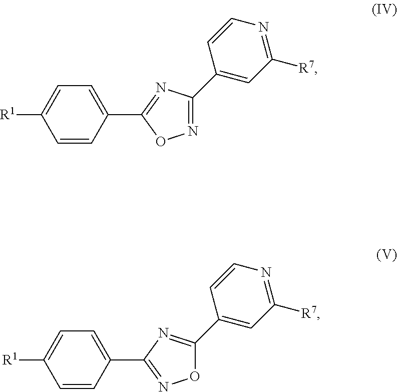
or a pharmaceutically acceptable salt thereof;
wherein,
R¹ is —CH₂NH₂, —CH₂NHCH₃, or —CH₂CH₂NH₂; and
R⁷ is Ar or OAr, wherein Ar is: 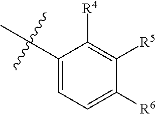
wherein,
R⁴ is H, CH₂(halo), or NO₂;
R⁵ is H or halo; and
R⁶ is H, halo, CF₃, —C(═O)CH₃, or —C≡CH.

12. The compound of claim 11 wherein R¹ is —CH₂NH₂.

13. The compound of claim 11 wherein R⁷ is 4-(trifluoromethyl)phenyl or oxy-4-(trifluoromethyl)phenyl.

14. The compound of claim 1 wherein the compound is: 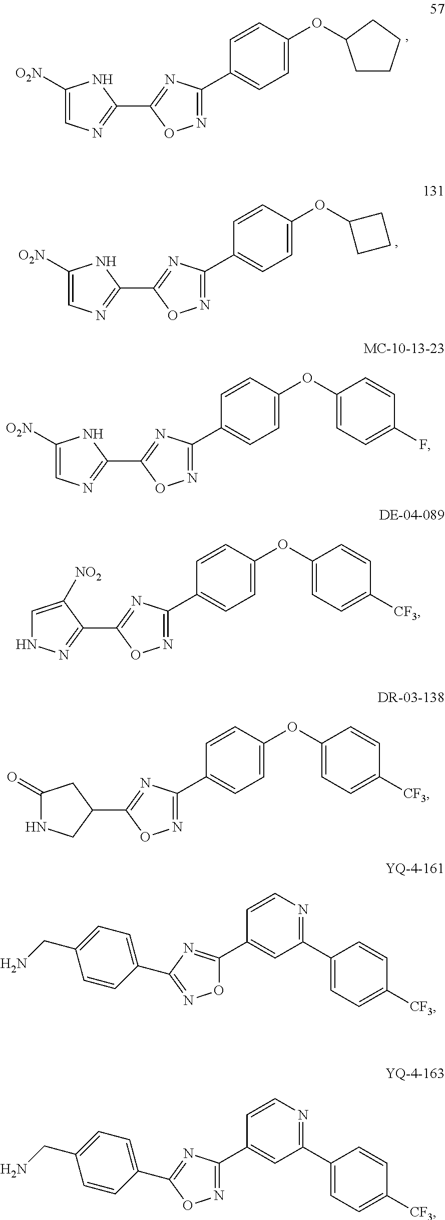 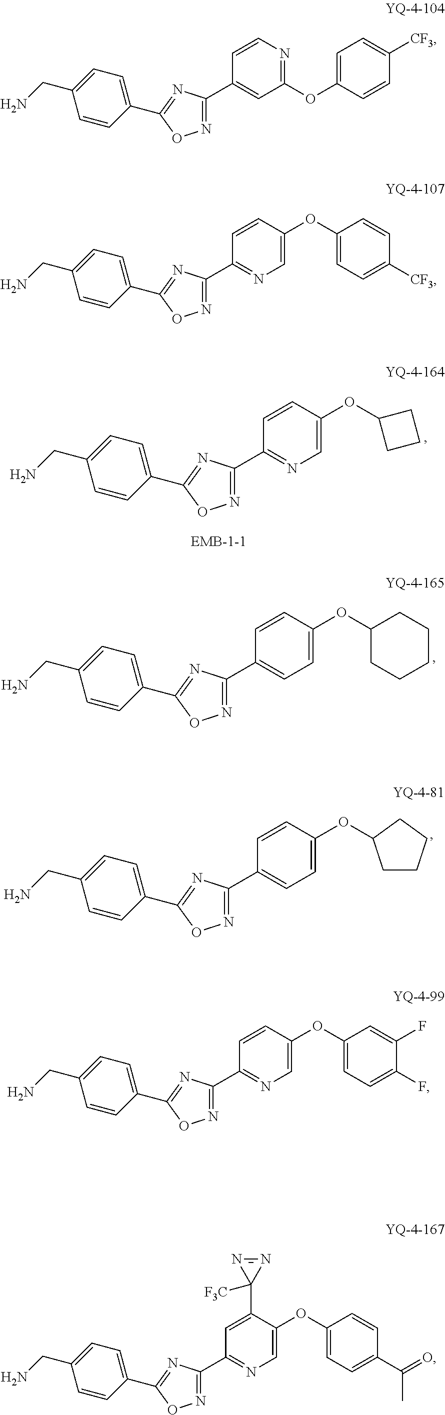 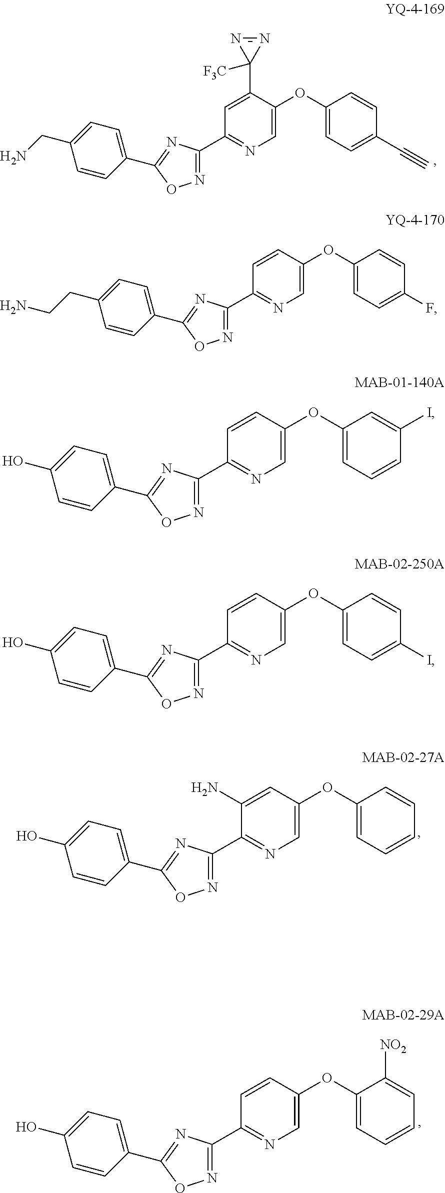 [SMILES: C#Cc1ccc(Oc2cnc(-c3noc(-c4ccc(CN)cc4)n3)cc2C2(C(F)(F)F)N=N2)cc1 ; NCCc1ccc(-c2nc(-c3ccc(Oc4ccc(F)cc4)cn3)no2)cc1 ; Oc1ccc(-c2nc(-c3ccc(Oc4cccc(I)c4)cn3)no2)cc1 ; Oc1ccc(-c2nc(-c3ccc(Oc4ccc(I)cc4)cn3)no2)cc1 ; Nc1cc(Oc2ccccc2)cnc1-c1noc(-c2ccc(O)cc2)n1 ; O=[N+]([O-])c1ccccc1Oc1ccc(-c2noc(-c3ccc(O)cc3)n2)nc1] 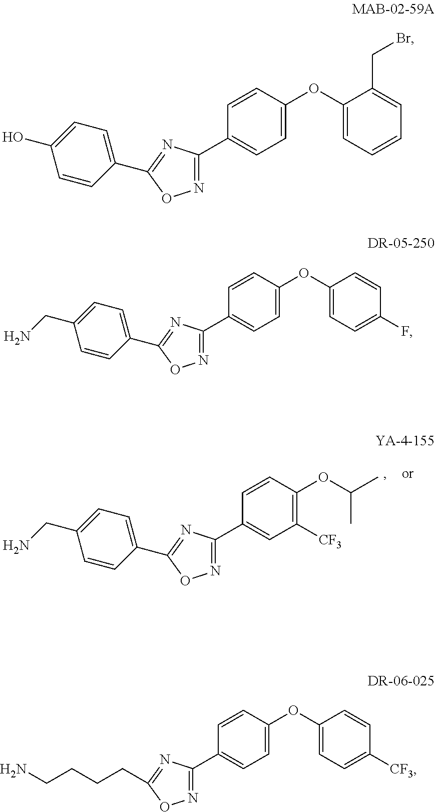 [SMILES: Oc1ccc(-c2nc(-c3ccc(Oc4ccccc4CBr)cc3)no2)cc1 ; NCc1ccc(-c2nc(-c3ccc(Oc4ccc(F)cc4)cc3)no2)cc1 ; CC(C)Oc1ccc(-c2noc(-c3ccc(CN)cc3)n2)cc1C(F)(F)F ; NCCCCc1nc(-c2ccc(Oc3ccc(C(F)(F)F)cc3)cc2)no1]
or a pharmaceutically acceptable salt thereof.

15. A method for treating a Clostridioides difficile infection (CDI) comprising administering to a subject having a CDI a therapeutically effective dose of a compound of claim 1, wherein the compound inhibits growth of a Clostridioides difficile vegetative cell or germination of a Clostridioides difficile spore that is present in the CDI and the subject is thereby treated.

16. The method of claim 15 wherein the Clostridioides difficile spore is in a vegetative state.

17. The method of claim 15 wherein the compound selectively inhibits germination of a Clostridioides difficile spore by damaging the cell wall of the spore.

18. The method of claim 15 wherein the compound is inactive toward inhibiting microbiome gut bacteria of the subject, or wherein the compound is inactive toward inhibiting Gram-negative bacteria.

19. The method of claim 15 wherein the compound is administered, concurrently or sequentially, with an antibiotic capable of treating a CDI.

20. The method of claim 15 wherein the compound is 3-(4-(cyclopentyloxy)phenyl)-5-(5-nitro-1H-imidazol-2-yl)-1,2,4-oxadiazole (57).
